(19) United States
(12) Patent Application Publication
(10) Pub. No.: US 2020/0149063 A1
(43) Pub. Date: May 14, 2020

(54) METHODS FOR GENDER DETERMINATION AND SELECTION OF AVIAN EMBRYOS IN UNHATCHED EGGS

(71) Applicant: EGGXYT LTD., Jerusalem (IL)

(72) Inventors: Daniel OFFEN, Kfar Haroe (IL); Yehuda ELRAM, Tel Aviv (IL)

(21) Appl. No.: 16/729,921

(22) Filed: Dec. 30, 2019

Related U.S. Application Data
(63) Continuation-in-part of application No. 15/996,045, filed on Jun. 1, 2018.
Application No. 15/996,045 is a continuation-in-part of application No. PCT/IL2016/051291, filed on Dec. 1, 2016.
Application No. PCT/IL2016/051291 is a continuation-in-part of application No. 16/616,858.
Application No. 16/616,858 is a continuation-in-part of application No. 62/785,756, filed on Dec. 28, 2018.
(60) Provisional application No. 62/262,409, filed on Dec. 3, 2015.
(60) Provisional application No. 62/510,921, filed on May 25, 2017.

(51) Int. Cl. C12N 15/85; C12Q 1/6879; C12Q 1/6897; A01K 67/027

(52) U.S. Cl. CPC C12N 15/8509; C12Q 1/6879; C12Q 1/6897; A01K 2227/30; C12Q 2600/124; A01K 2217/05; A01K 67/0275

(57) ABSTRACT

The present invention, in some embodiments thereof, is directed to a method for selecting a gender of an avian fertilized unhatched egg obtained from the crossing of a transgenic male avian subject and a transgenic female avian subject. Further provided is a kit for preparing a transgenic avian subject.

### CROSS-REFERENCE TO RELATED APPLICATIONS

This application is a continuation-in-part of U.S. patent application Ser. No. 15/996,045, filed Jun. 1, 2018, titled “METHODS FOR GENDER DETERMINATION OF AVIAN EMBRYOS IN UNHATCHED EGGS AND MEANS THEREOF”, which is a continuation-in-part of International Patent Application No. PCT/IL2016/051291, filed Dec. 1, 2016, titled “METHODS FOR GENDER DETERMINATION OF AVIAN EMBRYOS IN UNHATCHED EGGS AND MEANS THEREOF”, which claims the benefit of priority of U.S. Provisional Patent Application No. 62/262,409, filed Dec. 3, 2015, titled “A METHOD FOR GENDER SELECTION OF UNHATCHED EGGS BY BIOLUMINESCENCE”.

This application is also a continuation-in-part of U.S. patent application Ser. No. 16/616,858, filed Nov. 25, 2019, titled “METHODS FOR GENDER DETERMINATION OF AVIAN EMBRYOS IN UNHATCHED EGGS AND MEANS THEREOF”, which is a national phase of International Patent Application No. PCT/IL2018/050573, filed May 24, 2018, titled “METHODS FOR GENDER DETERMINATION OF AVIAN EMBRYOS IN UNHATCHED EGGS AND MEANS THEREOF”, which claims the benefit of priority of U.S. Provisional Patent Application No. 62/510,921, filed May 25, 2017, titled “METHODS FOR GENDER DETERMINATION OF AVIAN EMBRYOS IN UNHATCHED EGGS AND MEANS THEREOF”.

This application is also a continuation-in-part of U.S. Provisional Patent Application No. 62/785,756, filed Dec. 28, 2018, titled “METHODS FOR GENDER SELECTION OF AVIAN IN UNHATCHED EGGS AND MEANS THEREOF”.

The contents of all the above applications are incorporated herein by reference in their entirety.

### FIELD OF THE INVENTION

The present invention relates to methods of gender determination and identification in avian subjects. More specifically, the invention provides non-invasive methods and transgenic avian animals for gender determination and selection of embryos in unhatched avian eggs.

### BACKGROUND OF THE INVENTION

Each day, billions of male chicks are being terminated via suffocation or grinding since they are not useful for laying eggs or to be bread for meat. The ability to determine the sex of the embryo before hatching and selecting females for subsequent rearing is therefore of high importance both ethically and financially. In the chicken—the genetic make-up of the sex chromosomes is ZZ for males and ZW (or WZ) for females. Meaning the W chromosome determines the gender of the female. This is unlike humans, in which it is the Y from the father that determines the male gender.

As mentioned above, in all commercial types of birds intended for breeding, laying, or meat production, there is a need to determine fertility and the sex of the embryo. Therefore, there are great economic returns; in energy saving, biosecurity risk reduction, garbage disposal, sexing labor costs and sexing errors, culling costs and disposal, and animal welfare.

Effective and non-invasive methods for sex identification and subsequent selection during the egg stage, prior to the hatching of the chick are currently not available. There is therefore a long-felt need for a method enabling accurate and safe sex identification of the embryos in unhatched eggs and subsequent selection for rearing.

### SUMMARY OF THE INVENTION

The following embodiments and aspects thereof are described and illustrated in conjunction with systems, tools and methods which are meant to be exemplary and illustrative, not limiting in scope.

The present invention, in some embodiments thereof, utilizes transgenic avian subjects so as to select a desired gender. In some embodiments, the present method enables the production of male-free flocks. The present method, in some embodiments thereof, is based in part, on a breeding pair comprising complementing CRISPR-Cas components expressed during an early embryonic stage, which in turn enables to knock-out a gene vital for cell survival and/or development only after fertilization occurs, as early as 5 days post egg laying, the latest. The present method, in some embodiments thereof, further provides an initial selection step according to which at least one reporter gene, e.g., a fluorescent protein, is further integrated into the genome of the mother, or of the father.

The present invention, in some embodiments thereof, provides a highly efficient and selective 2-step methodology so as to eliminate non-fertilized eggs, eggs comprising male embryos, or both, with great proficiency, as early as day 5post egg laying, the latest (e.g., before candling).

According to a first aspect, there is provided a method for selecting a gender of an avian fertilized unhatched egg, the method comprising: (a) providing a transgenic male avian subject comprises at least one exogenous gene comprising a first nucleic acid sequence integrated into at least one position or location in both of the male avian gender chromosomes Z; (b) providing a transgenic female avian subject comprising at least one exogenous gene comprising a second nucleic acid sequence integrated into at least one position or location in gender chromosome Z; wherein the first nucleic acid sequence or the second nucleic acid sequence is operably linked to an embryonal promoter; such that a fertilized unhatched egg from the transgenic female avian subject and the transgenic male avian subject comprising the combination of the first nucleic acid sequence and the second nucleic acid, results in targeting at least one avian vital gene, thereby selecting a gender of the avian fertilized unhatched egg.

According to another aspect, there is provided a kit comprising: (a) at least one first nucleic acid sequence encoding any one of at least one Cas protein and at least one gRNA; (b) a second nucleic acid sequence, wherein when the first nucleic acid sequence encodes at least one Cas protein the second nucleic acid encodes at least one gRNA, wherein when the first nucleic acid sequence encodes at least one gRNA the second nucleic acid encodes at least one Cas protein, and wherein the combination of the at least one Cas protein and the at least one gRNA targets at least one vital avian gene; (c) optionally a third nucleic acid sequence, a fourth nucleic acid sequence, or both, wherein any one of the third nucleic acid sequence and the fourth nucleic acid sequence encodes at least one reporter gene having an excitation wavelength ranging from 500 to 650 nm and an emission wavelength ranging from 550 to 650 nm; and (d) instructions for the preparation of any one of: (i) at least one transgenic male avian subject comprising the first nucleic acid sequence and optionally the fourth nucleic acid integrated into at least one locus within both gender chromosomes Z of the male avian subject, (ii) at least one transgenic female avian subject comprising the second nucleic acid sequence and optionally the third nucleic acid, wherein the second nucleic acid is integrated into at least one locus within gender chromosome Z of the female avian subject and optionally the third nucleic acid is integrated into at least one locus within gender chromosome Z or W of the female avian subject, and (i) and (ii).

According to another aspect, there is provided a transgenic male avian subject prepared according to the herein disclosed kit.

According to another aspect, there is provided a transgenic female avian subject prepared according to the herein disclosed kit.

In some embodiments, the embryonal promoter initiates the expression of the first nucleic acid sequence or the second nucleic acid sequence on day 1 to day 5 post egg laying.

In some embodiments, any one of the first nucleic acid sequence and the second nucleic acid sequence is operably linked to an embryonal promoter.

In some embodiments, any one of: (a) the transgenic female avian further comprises a third nucleic acid sequence encoding at least one reporter gene encoding a protein product having an excitation wavelength ranging from 500 to 650 nm and an emission wavelength ranging from 550 to 650 nm, wherein the third nucleic acid is integrated into at least one position or location in gender chromosome W or Z; (b) the transgenic male avian further comprises a fourth nucleic acid sequence encoding at least one reporter gene encoding a protein product having an excitation wavelength ranging from 500 to 650 nm and an emission wavelength ranging from 550 to 650 nm, wherein the fourth nucleic acid is integrated into at least one position or location in both of gender chromosomes Z, and (a) and (b).

In some embodiments, the at least one reporter gene encodes a fluorescent protein.

In some embodiments, the at least one reporter gene encodes a red fluorescent protein (RFP).

In some embodiments, the method further comprises a step of detecting at least one detectable signal of the protein product in the fertilized unhatched egg, wherein detection of the at least one detectable signal is indicative of the expression of the reporter gene in the fertilized egg, thereby determining that the fertilized unhatched egg comprises any embryo selected from the group consisting of: a male embryo, a female embryo, and both.

In some embodiments, the step of detecting the at least one detectable signal is performed on day two (2) post egg laying, at most.

In some embodiments, detecting comprises subjecting the fertilized unhatched egg to a light source.

In some embodiments, any one of the first nucleic acid sequence and the second nucleic acid sequence encodes at least one guide RNA (gRNA) or at least one CRISPR associated protein.

In some embodiments, any one of the first nucleic acid sequence and the second nucleic acid sequence is integrated into the gender chromosome Z of any one of the female transgenic avian subject and the male avian subject by contacting or co-transfecting at least one cell of any one of the female transgenic avian subject and the male transgenic avian subject, with any one of: at least one Cas protein or at least one nucleic acid sequence encoding thereof, and at least one gRNA or at least one nucleic acid sequence encoding thereof, wherein the gRNA targets at least one protospacer within the at least one gender chromosome Z.

In some embodiments, any one of the first nucleic acid sequence and the second nucleic acid sequence is integrated into at least one site in locus 42172748-42177748 of the gender chromosome Z.

In some embodiments, avian vital gene is crucial for at least one trait selected from a group consisting of: cell vitality, cell mitosis, cell metabolism, cell differentiation, DNA polymerization, RNA transcription, protein translation and housekeeping genes, and any combination thereof.

In some embodiments, the transgenic female avian subject further comprises the fourth nucleic acid sequence.

In some embodiments, the transgenic female avian subject further comprises the third nucleic acid sequence.

In some embodiments, the kit further comprises instructions for breeding the at least one transgenic male avian subject with the at least one transgenic female avian subject so as to obtain a progeny.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this

### DETAILED DESCRIPTION

In addition to the exemplary aspects and embodiments described above, further aspects and embodiments will become apparent by study of the following detailed description.

### BRIEF DESCRIPTION OF THE FIGURES

FIG. 1 is a scheme of a non-limiting design for breeding or crossing a transgenic female avian subject and a male transgenic avian subject so as to obtain male-free flocks. The transgenic female avian subject comprises a wild type (WT) gender chromosome W and a gender chromosome Z comprising a guide RNA (gRNA) targeting a critical gene for survival and/development (*). The transgenic male avian subject comprises a Cas protein encoding sequence under the regulation of an embryonal promoter (EP) on both of its gender chromosomes Z. Any male offspring obtained from the disclosed breeding/crossing bears Cas-gRNA complex which in turn knocks out the vital gene. Accordingly, only female embryos develop, and male-free flocks are achieved.

FIG. 2 is a scheme of a non-limiting design for breeding or crossing a transgenic female avian subject and a male transgenic avian subject so as to obtain male-free flocks. The transgenic female avian subject comprises a wild type (WT) gender chromosome W and a gender chromosome Z comprising a Cas protein encoding sequence under the regulation of an embryonal promoter (EP). The transgenic male avian subject comprises a guide RNA (gRNA) targeting a critical gene for survival and/development (*) on both of its gender chromosomes Z. Any male offspring obtained from the disclosed breeding/crossing bears Cas-gRNA complex which in turn knocks out the vital gene. Accordingly, only female embryos develop, and male-free flocks are achieved.

FIG. 3 is a scheme of a non-limiting design for breeding or crossing a transgenic female avian subject and a male transgenic avian subject so as to obtain male-free flocks. The transgenic female avian subject comprises a wild type (WT) gender chromosome W and a gender chromosome Z comprising an exogenous reporter gene (RG) and guide RNA (gRNA) targeting a critical gene for survival and/development (*). The transgenic male avian subject comprises a Cas protein encoding sequence under the regulation of an embryonal promoter (EP), on both of its gender chromosomes Z. Any male offspring obtained from the disclosed breeding/crossing bears a copy of the RG (black egg), therefore detection of which enables the disposal of the vast majority of male embryos, as early as 24-48 hours post egg laying. In the case that RG expression is hampered, undetected, or any equivalent thereof (e.g., “evaders”) all male embryos comprise a Cas-gRNA complex which in turn knocks out the vital gene. Accordingly, only female embryos develop by day 5 post egg laying, and male-free flocks are achieved.

FIG. 4 is a scheme of a non-limiting design for breeding or crossing a transgenic female avian subject and a male transgenic avian subject so as to obtain male-free flocks. The transgenic female avian subject comprises a wild type (WT) gender chromosome W and a gender chromosome Z comprising an exogenous reporter gene (RG) and a Cas protein encoding sequence under the regulation of an embryonal promoter (EP). The transgenic male avian subject comprises a guide RNA (gRNA) targeting a critical gene for survival and/development (*), on both of its gender chromosomes Z. Any male offspring obtained from the disclosed breeding/crossing bears a copy of the RG (black egg), therefore detection of which enables the disposal of the vast majority of male embryos, as early as 24-48 hours post egg laying. In the case that RG expression is hampered, undetected, or any equivalent thereof (e.g., “evaders”) all male embryos comprise a Cas-gRNA complex which in turn knocks out the vital gene. Accordingly, only female embryos develop by day 5 post egg laying, and male-free flocks are achieved.

FIG. 5 is a scheme of a non-limiting design for breeding or crossing a transgenic female avian subject and a male transgenic avian subject so as to obtain male-free flocks. The transgenic female avian subject comprises a gender chromosome W comprising an exogenous reporter gene (RG) and a gender chromosome Z comprising a Cas protein encoding sequence under the regulation of an embryonal promoter (EP). The transgenic male avian subject comprises a guide RNA (gRNA) targeting a critical gene for survival and/development (*), on both of its gender chromosomes Z. Any female offspring obtained from the disclosed breeding/crossing bears a copy of the RG (black egg), therefore detection of which (“labeled eggs”) enables the determination that the egg is fertilized and comprises a female embryo. Non “labeled eggs” comprise predominantly male embryos, which can be disposed as early as 24-48 hours post egg laying. At any rate, all the male embryos comprise a Cas-gRNA complex which in turn knocks out the vital gene. Accordingly, only female embryos develop by day 5 post egg laying, and male-free flocks are achieved. Alternatively, the transgenic female avian subject can comprise the gRNA* (and not the Cas protein encoding sequence) when the transgenic male avian subject comprises the Cas protein encoding sequence (and not the gRNA*) while resulting in male-free flocks as described above.

FIG. 6 is a scheme of a non-limiting design for breeding or crossing a transgenic female avian subject and a male transgenic avian subject so as to obtain male-free flocks. The transgenic female avian subject comprises a wild type (WT) gender chromosome W and a gender chromosome Z comprising a Cas protein encoding sequence under the regulation of an embryonal promoter (EP). The transgenic male avian subject comprises a guide RNA (gRNA) targeting a critical gene for survival and/development (*), and an exogenous reporter gene (RG), on both of its gender chromosomes Z. Any successful fertilization results in an offspring bearing a copy of the RG, therefore the detection of which serves as a “fertilization cut-off”, which enables the disposal of any unfertilized egg, as early as 24-48 hours post egg laying. Only male embryos comprise a Cas-gRNA complex which in turn knocks out the vital gene. Accordingly, only female embryos develop by day 5 post egg laying, and male-free flocks are achieved. Alternatively, the transgenic female avian subject can comprise the gRNA* (and not the Cas protein encoding sequence) when the transgenic male avian subject comprises the Cas protein encoding sequence (and not the gRNA*) while resulting in male-free flocks as described above.

FIG. 7 is a scheme of a non-limiting design for breeding or crossing a transgenic female avian subject and a male transgenic avian subject so as to obtain male-free flocks. The transgenic male avian subject comprises a guide RNA (gRNA) targeting a critical gene for survival and/development (*), and a first exogenous reporter gene (RG1), on both of its gender chromosomes Z. The transgenic female avian subject comprises a gender chromosome W comprising a second exogenous reporter gene (RG2) and a gender chromosome Z comprising a Cas protein encoding sequence under the regulation of an embryonal promoter (EP). Only eggs successfully fertilized so as to yield a female offspring bear a combination of RG1 and RG2, therefore the detection of which serves as a “female fertilization cut-off”, which enables the disposal of any unfertilized egg, any egg comprising a male embryo, or both, as early as 24-48 hours post egg laying. At any rate, male embryos comprise a Cas-gRNA complex which in turn knocks out the vital gene. Accordingly, only female embryos develop by day 5 post egg laying, and male-free flocks are achieved. Alternatively, the transgenic female avian subject can comprise the gRNA* (and not the Cas protein encoding sequence) when the transgenic male avian subject comprises the Cas protein encoding sequence (and not the gRNA*) while resulting in male-free flocks as described above.

FIGS. 8A-8B are fluorescent micrographs showing fluorescently labeled chick fibroblasts. The fibroblasts were genetically-edited so as to integrate the reporter gene, e.g., red fluorescent protein (RFP) in a locus of the gender chromosome Z.

FIGS. 9A-9B are images showing that luciferase reporter gene signal is formed in a fertilized egg and penetrates the eggshell. Ear (9A) and tail (9B), excised from luciferase expressing transgenic mice, were incorporated into a fertilized carrying a 10 days old chicken embryo. Luciferin was subsequently injected to induce bioluminescence. Images were taken 10 minutes thereafter using the bio-space photon Imager (Bio space lab, USA).

FIGS. 10A-10B are fluorescent micrographs showing that GFP reporter gene signal is not detectable through the eggshell. Tail from GFP-expressing transgenic mice were incorporated into chicken embryo (10 days) or placed outside of the shell. Only tail placed outside of the eggshell (10A) was observed with GFP fluorescence, whereas no signal was detected when placed inside the egg (10B). Images were taken after 5 minutes thereafter using the Maestro 2.2 Imager (Cambridge Research & Instrumentation, Inc. USA).

FIG. 11 is a graph showing the fluorescence intensity using a green laser (532 nm) with or without dir. The fluorescence intensity [Pout] that was received on the detector, when a complete egg was subjected to green laser with or without dir, and the intensity is represented as a function of the light source intensity [Pin].

FIG. 12 is a graph showing the fluorescence intensity using a red laser (632.8 nm) with or without dir. The fluorescence intensity [Pout] that was received on the detector, when a complete egg was subjected to red laser with or without dir, and the intensity is represented as a function of the light source intensity [Pin].

FIG. 13 is a graph showing measurements of fluorescence intensity of eggs injected with different concentrations of RFP-expressing cells using a green laser and the red filter. The fluorescence intensity [Pout] that was received on the detector is represented as a function of the light source intensity [Pin].

FIG. 14 is a graph showing measurements of fluorescence intensity of eggs injected with GFP-expressing cells. The figure shows fluorescence intensity of eggs injected with 30,000 GFP-expressing cells suspended with PBS or Glycerol using the Blue laser. The fluorescence intensity [Pout] that was received on the detector is represented as a function of the light source intensity [Pin].

FIG. 15 is a graph showing comparative fluorescence measurements of RFP vs. GFP. The figure shows ratio between the fluorescence protein intensity of either GFP-expressing cells or RFP-expressing cells and the auto fluorescence intensity. Parameter R describing the ratio between fluorescence and auto fluorescence is represented as a function of the light source intensity [Pin].

FIG. 16 is a fluorescent micrograph showing incorporation of RFP into a female's Z chromosome. The figure shows RFP transfected female chicken cell line with pDsRed with chicken ChZ Left & Right arms and CMV-hspCas9-H1-gRNA.

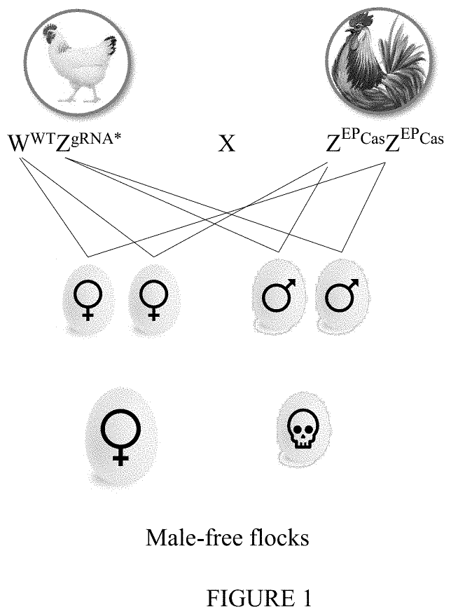

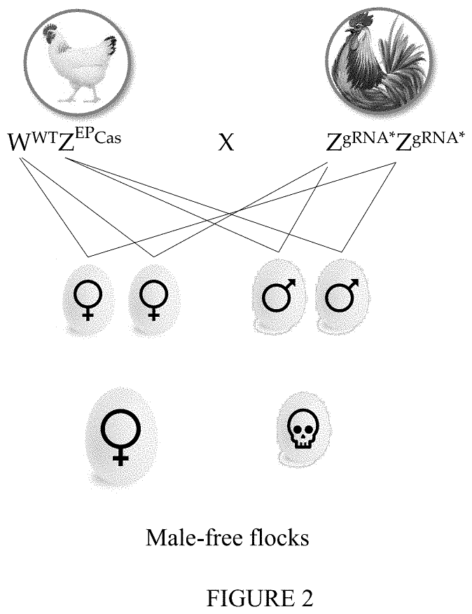

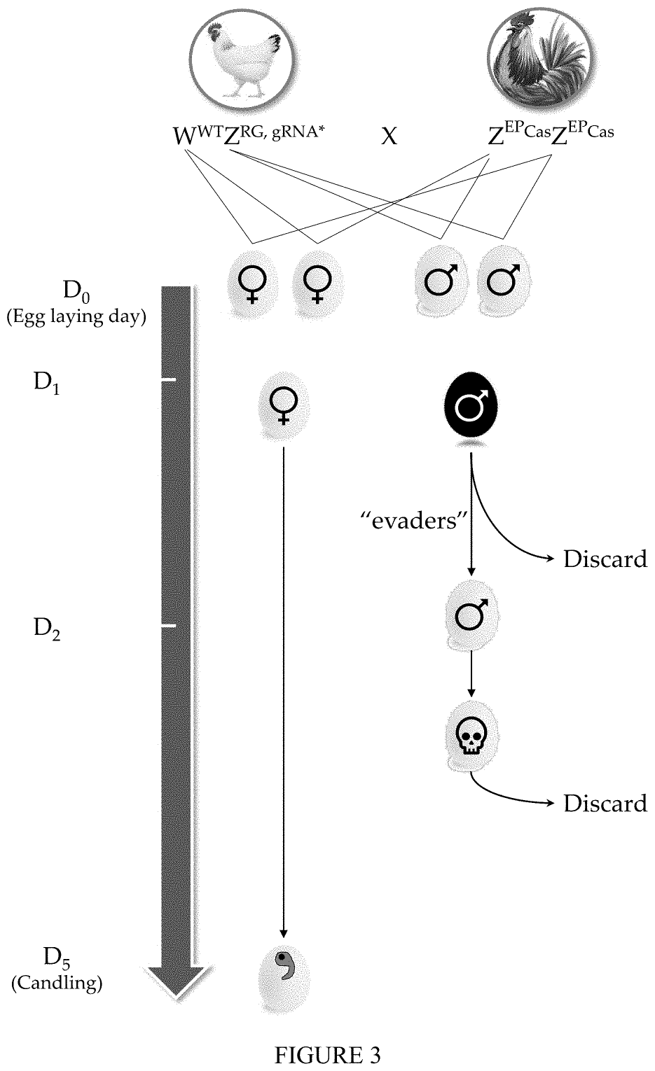

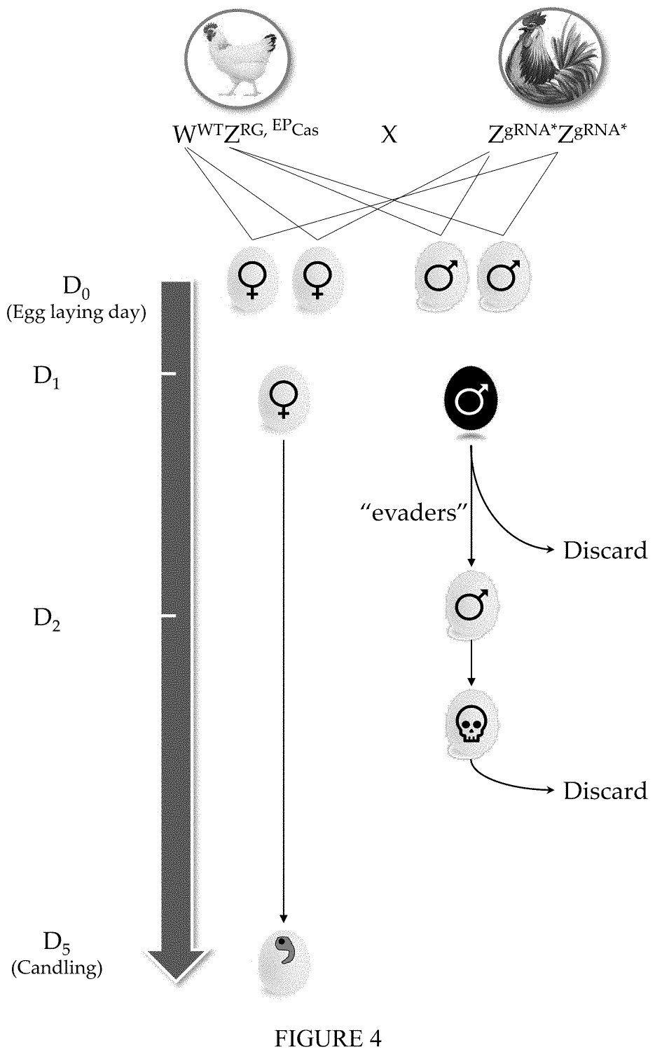

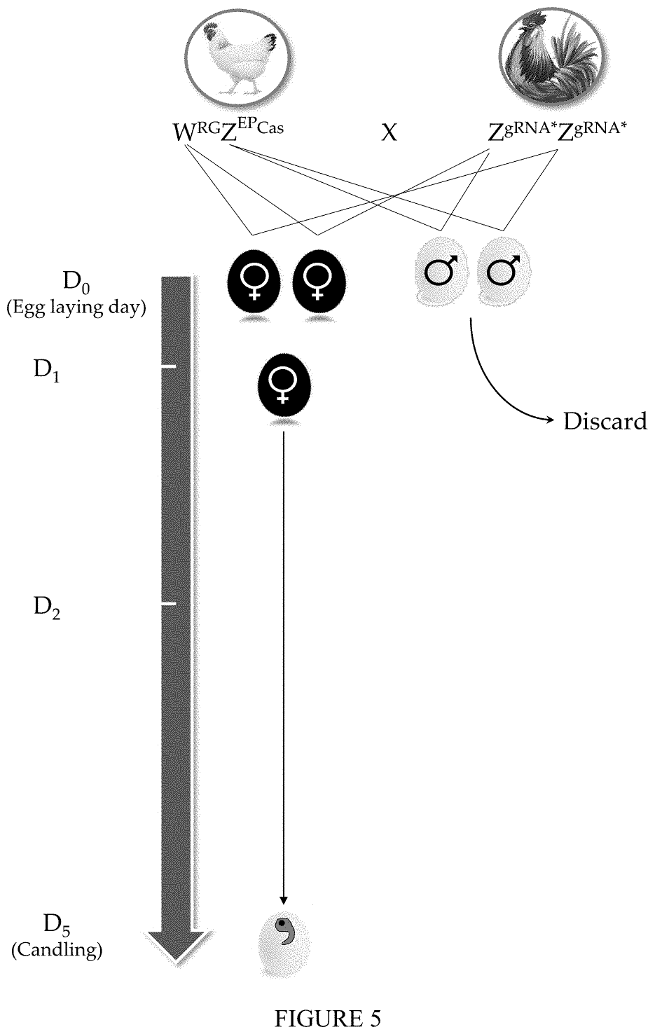

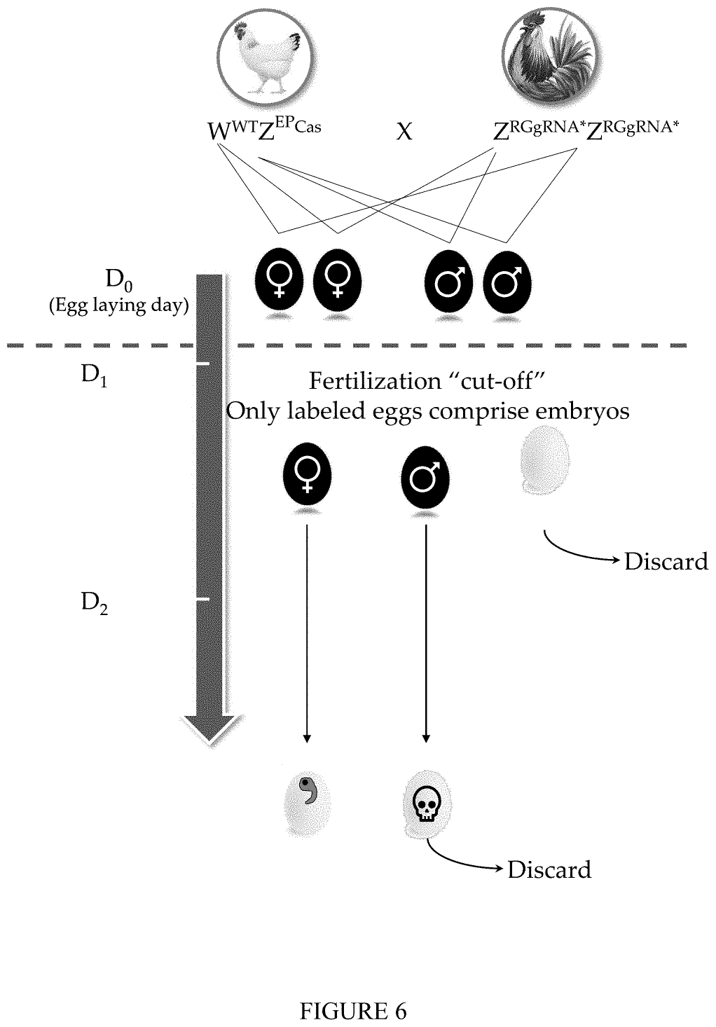

### DETAILED DESCRIPTION OF THE INVENTION

According to some embodiments, the present invention is directed to a method for selecting a gender of an avian fertilized unhatched egg e.g., male-free flocks.

In some embodiments, the method comprising: (a) providing a transgenic male avian subject comprising at least one exogenous gene comprising a first nucleic acid sequence integrated into at least one position or location in both of the male avian gender chromosomes Z; (b) providing a transgenic female avian subject comprising at least one exogenous gene comprising a second nucleic acid sequence integrated into at least one position or location in gender chromosome Z; wherein the first nucleic acid sequence or the second nucleic acid sequence is operably linked to an embryonal promoter; such that a fertilized unhatched egg from the transgenic female avian subject and the transgenic male avian subject comprising the combination of the first nucleic acid sequence and the second nucleic acid, results in targeting at least one avian vital gene, thereby selecting a gender of the avian fertilized unhatched egg.

In some embodiments, a transgenic female avian further comprises a third nucleic acid sequence encoding at least one reporter gene encoding a protein product. In some embodiments, a transgenic male avian further comprises a third nucleic acid sequence encoding at least one reporter gene encoding a protein product. In some embodiments, a transgenic female avian further comprises a third nucleic acid sequence encoding at least one first reporter gene encoding a protein product and a transgenic male avian further comprises a fourth nucleic acid sequence encoding at least one second reporter gene encoding a protein product, or vice versa.

As used herein, the term “operably linked” is intended to mean that the nucleotide sequence of any one of the herein disclosed exogenous genes is linked to a regulatory element or elements, e.g., the promoter, in a manner that allows for expression of the nucleotide sequence (e.g., in an in vitro transcription/translation system or in a host cell when a vector is introduced into the host cell).

In some embodiments, there is provided a transgenic male avian subject comprising at least one exogenous gene comprising a first nucleic acid sequence of the invention integrated into at least one position or location in both of the transgenic male avian gender chromosomes Z. In some embodiments, the transgenic male avian subject further comprises a fourth nucleic acid encoding at least one second reporter gene.

In some embodiments, there is provided a transgenic female avian subject comprising at least one exogenous gene comprising a second nucleic acid sequence integrated into at least one position or location in gender chromosome Z or W, of the transgenic female avian subject. In some embodiments, the transgenic female avian subject further comprises a third nucleic acid encoding at least one first reporter gene.

In some embodiments, any one of the herein disclosed first nucleic acid sequence, second nucleic acid sequence, third nucleic acid sequence, and fourth nucleic acid sequence is under the control of an embryonic promoter, thereby limiting the expression of the exogenous gene to a specific embryonal stage or timing, with no expression in the adult chick, or in specific embryonic stages. In some embodiments, any one of the herein disclosed exogenous genes may be used and expressed only at an embryonal stage, for example, for diagnostic purposes, or driving expression of an exogenous gene or a transgene in a particular embryonic developmental stage.

In one embodiment, any one of the third nucleic acid sequence and the fourth nucleic acid sequence comprising at least one first reporter gene, the at least one second reporter gene, or both, can be under the control of a non-embryonic promoter, as described herein, e.g., a constitutive promoter, an inducible promoter, etc.

In some embodiments, when the first nucleic acid sequence or the second nucleic sequence encodes a guide RNA (gRNA) as disclosed hereinbelow, the first nucleic acid sequence or the second nucleic sequence can be under the control of a non-embryonic promoter. In some embodiments, a non-embryonic promoter comprises or is a constitutive promoter. In some embodiments, a non-embryonic promoter comprises or is an inducible promoter. In some embodiments, the gRNA is under the control of a pol III promoter. In some embodiments, a pol III promoter is selected from U6 or U3.

As used herein, the term “promoter” refers to a particular region of the DNA that has the ability to control or drive the expression of a nucleic acid, e.g., a gene, which is placed downstream to the promoter. In some embodiments, the promoter is gender-specific in chicks.

In some embodiments, embryonal promoter initiates, starts, enables, drives, propagates, enhances, or any molecular equivalent thereof, the expression of any one of the first nucleic acid sequence and the second nucleic acid sequence on day 1 to day 2 post egg laying, on day 1 to day 3 post egg laying, on day 1 to day 4 post egg laying, on day 1 to day 5 post egg laying, on day 2 to day 3 post egg laying, on day 2 to day 4 post egg laying, on day 2 to day 5 post egg laying, on day 3 to day 4 post egg laying, on day 3 to day 5 post egg laying, or on day 4 to day 5 post egg laying. Each possibility represents a separate embodiment of the invention.

In some embodiments, the embryonal promoter is any one of: an early stage embryonic promoter, spermatogenesis-related promoter, skeletal muscle differentiation promoter, and gonad specific promoter. In some embodiments, the embryonic promoter is selected from: PouV promoter (Oct4 homolog; chr17:826223-829223), Sox 2 promoter (and/or other SoxB1 family of promoter; chr9:17026706-17029706), Nodal promoter (chr22:2571106-2574106), Notch promoter (chr17:8106140-8109143), Nanog promoter (chr1:75596024-75599025), FGF3 promoter (chr5:17877619-17880620), FGF8 promoter (chr6:23666486-23669486), KLF4 promoter (chrZ:55792226-55795226), KLF5 promoter (chr1:157456439-157459439), Piwi promoter (chr15:3647097-3650097), miR206 promoter (chr3:107584598-107587598), MyoD promoter (chr5: 12392624-12395624), cENS-1 promoter (chr2 :36190015-36193015), and LRH1 (e.g., NR5A2) promoter (chr8:2060775-2063776). In some embodiments, the embryonic promoter location as referenced herein, is based on the genome of the chicken Gallus.

In some embodiments, the embryonal promoter comprises or consists of a nucleic acid sequence comprising: 100 to 500 bp, 250 to 1,000 bp, 500 to 3,100 bp, 700 to 2,500 bp, 200 to 2,500 bp, 300 to 2,750 bp, or 150 to 4,000 bp. Each possibility represents a separate embodiment of the invention.

In some embodiments, the embryonal promoter comprises or consists of a nucleic acid sequence set forth in any one of: SEQ ID Nos: 1-14.

In some embodiments, the embryonal promoter comprises or consists of a nucleic acid sequence comprising: 100 to 500 bp, 250 to 1,000 bp, 500 to 3,100 bp, 700 to 2,500 bp, 200 to 2,500 bp, 300 to 2,750 bp, or 150 to 4,000 bp, wherein the nucleic acid sequence is set forth in any one of: SEQ ID Nos: 1-14. Each possibility represents a separate embodiment of the invention.

As used herein, the term “vital gene” refers to any gene which encodes a product, e.g., a protein, or a functional RNA molecule, that is mandatory or essential so as to provide a viable and/or functional cell. In some embodiments, reducing, inhibiting, eliminating, or any equivalent thereof, of the activity the product results in apoptosis, necrosis, cell death, reduced survival, reduced fitness, impaired functional activity, or any combination thereof, of a cell or an organism comprising thereof. As used herein, the term “vital gene” encompasses any gene that inhibiting, reducing, blocking, eliminating, omitting, or any equivalent thereof, of the activity of the product of the gene at any time from egg laying to 5 days post laying at most, results in apoptosis, necrosis, cell death, reduced survival, reduced fitness, impaired functional activity, or any combination thereof, of a cell or an organism comprising thereof. In some embodiments, eliminating the activity comprises knocking the gene out of the genome of the cell. In some embodiments, eliminating the activity comprises knocking-down the RNA or mRNA encoded from the gene, e.g., by RNA interference (RNAi). In some embodiments, eliminating the activity comprises integrating a nucleic acid within the gene in the genome, wherein the integration creates a frameshift in the gene thereby disrupting the RNA or the open reading frame, and thereby disrupting the protein product translated from the mRNA encoded by the gene.

Methods for determining reduced cell functionality, viability or survival are common and would be apparent to one of ordinary skill in the art. Non-limiting examples include, but are not limited to, viability stains (such as acridine orange-Ethidium bromide), metabolic assays, cell proliferation (MTT, XTT), TUNEL assay, and others.

In some embodiments, a vital gene is selected from genes which are involved, promote, initiate, propagate, or any equivalent thereof, in: cell vitality, cell mitosis, cell metabolism, cell differentiation, DNA polymerization, RNA transcription, protein translation and/or modification, housekeeping genes, or any combination thereof.

A non-limiting example of a vital gene suitable for the methods and kits of the invention may be selected from Table 1.

TABLE 1
Vital Genes to be Edited for Gender Selection

| Gene | | | | | On- |
|---|---|---|---|---|---|
| Description | Posi- | | | | Target |
| (Symbol) | tion | Strand | Sequence | PAM | Score |
| glyceraldehyde-3- | 484731 | − | TCTAGGGAAAGAGAGCACTG | AGG | 83.2* |
| phosphate | | | (SEQ ID NO: 15) | | |
| dehydrogenase | | | | | |
| (GAPDH) | | | | | |
| glyceraldehyde-3- | 482958 | − | GGTCTCGGCGCACCGCCGCG | GGG | 42.5* |
| phosphate | | | (SEQ ID NO: 16) | | |
| dehydrogenase | | | | | |
| (GAPDH) | | | | | |
| hypoxanthine | 3624549 | − | GTACACAGAGAGCTACAATG | TGG | 79.3 |
| phosphoribosyl- | | | (SEQ ID NO: 17) | | |
| transferase | | | | | |
| 1 (HPRT1) | | | | | |
| hypoxanthine | 3625305 | − | AATCATAGGATTGCTCAGGT | TGG | 58.1* |
| phosphoribosyl- | | | (SEQ ID NO: 18) | | |
| transferase | | | | | |
| 1 (HPRT1) | | | | | |
| RNA polymerase II | 2211134 | + | CAACATCCAGATCTACCCCG | TGG | 79.2 |
| subunit H (POLR2H) | | | (SEQ ID NO: 19) | | |
| RNA polymerase II | 11310803 | − | GGCGAGACATTAGAAGAACG | CGG | 77.1* |
| subunit F (POLR2F) | | | (SEQ ID NO: 20) | | |
| RNA polymerase II | 11311935 | + | AATGGCCTCAAGTTGCACCA | AGG | 0.5 |
| subunit F (POLR2F) | | | (SEQ ID NO: 21) | | |
| protein kinase AMP- | 266919 | − | GAAACTCCGGGTTAACCCAG | CGG | 78.6* |
| activated non- | | | (SEQ ID NO: 22) | | |
| catalytic subunit | | | | | |
| gamma 1 (PRKAG1) | | | | | |
| protein kinase AMP- | 266512 | + | GGGGGGGGGGGGGAAGGAAA | GGG | 34.7* |
| activated non- | | | (SEQ ID NO: 23) | | |
| catalytic subunit | | | | | |
| gamma 1 (PRKAG1) | | | | | |
| NADH:ubiquinone | 122 | − | ACTGAAAATGGGCTTCACCG | AGG | 78.2 |
| oxidoreductase | | | (SEQ ID NO: 24) | | |
| subunit A6 (NDUFA6) | | | | | |
| NADH:ubiquinone | 1785 | − | ACTCGGGAGGCAGAGGCAGG | CGG | 55.1* |
| oxidoreductase | | | (SEQ ID NO: 25) | | |
| subunit A6 (NDUFA6) | | | | | |
| cytochrome c oxidase | 1419 | − | GATTTCTTGAAAGAGAACAG | AGG | 75.4* |
| copper chaperone | | | (SEQ ID NO: 26) | | |
| (COX17) | | | | | |
| cytochrome c oxidase | 1073 | + | GAGAAGGGGGAAGAAAACTG | CGG | 67.8 |
| copper chaperone | | | (SEQ ID NO: 27) | | |
| (COX17) | | | | | |
| ADP-ribosylation | 28424737 | + | AGGCTTCAATGTGGAAACAG | TGG | 74.3 |
| factor 5 (ARF5) | | | (SEQ ID NO: 28) | | |
| ADP-ribosylation | 28423584 | + | GGTGGCGGCGGCGGCGGCGG | CGG | 20.2* |
| factor 5 (ARF5) | | | (SEQ ID NO: 29) | | |

Scores marked with an * in Table 1, indicate that they were computed using the model as disclosed hereinbelow without gene position information.

One skilled in the art would appreciate that genomic data of avian species is available online, such as for Gallus gallus, e.g., in https://benchling.com, comprising genome version Gallus 5. The skilled artisan would appreciate that on-target score is important so as to selected gRNA and PAM. Doench, Fusi et al. (2016) describes two models for scoring guides—one that includes the position of the cut within the translated gene and a simpler model that looks only at the guide sequence. Therefore, optimized score can be inferred from Doench, Fusi et al. (2016), wherein this score is optimized for 20 bp guides with NGG PAMs. To this end, the on-target score ranges from 0-100 (e.g., the higher the better). In case using the model that includes gene position in the scoring function is desired: the guide(s) cut must be within a translated region of the sequence, and all of the translations on the sequence must be in a single translation group.

Further, the skilled artisan would appreciate the importance of off-target score. The specificity score can be inferred from Hsu et al. (2013). To this end, the off-target score ranges from 0-100 (e.g., the higher the better).

The term “reporter gene” relates to a gene or a fragment thereof which encodes a polypeptide, a fragment thereof, or any portion thereof, whose expression can be detected in a variety of assays and wherein the level of the detected signal indicates the presence, the level, or both, of the reporter gene and/or a product thereof.

In some embodiments, the third nucleic acid or the fourth nucleic acid sequence is integrated into at least one position or location in gender chromosome Z. In some embodiments, the third nucleic acid or the fourth nucleic acid sequence is integrated into at least one position or location in gender chromosome W. In some embodiments, the third nucleic acid or the fourth nucleic is integrated into at least one position or location in gender chromosome W or Z of the transgenic female avian of the invention. In some embodiments, the third nucleic acid or the fourth nucleic acid sequence is integrated into at least one position or location in both gender chromosomes Z. In some embodiments, the third nucleic acid or the fourth nucleic acid sequence is integrated into at least one position or location in both gender chromosomes Z of the transgenic male avian of the invention.

As used herein, in some embodiments, the reporter gene encodes a fluorescent protein.

In some the protein product of the herein disclosed at least one reporter gene has an excitation wavelength ranging from 500 to 525 nm, 500 to 540 nm, 500 to 560 nm, 500 to 575 nm, 500 to 590 nm, 500 to 600 nm, 500 to 610 nm, 500 to 625 nm, 500 to 640 nm, or 500 to 650 nm. Each possibility represents a separate embodiment of the invention.

In some the protein product of the herein disclosed at least one reporter gene has an emission wavelength ranging from 550 to 570 nm, 550 to 590 nm, 550 to 605 nm, 550 to 615 nm, 550 to 625 nm, 550 to 640 nm, or 550 to 650 nm. Each possibility represents a separate embodiment of the invention.

In some the protein product of the herein disclosed at least one reporter gene has an excitation wavelength ranging from 500 to 525 nm, 500 to 540 nm, 500 to 560 nm, 500 to 575 nm, 500 to 590 nm, 500 to 600 nm, 500 to 610 nm, 500 to 625 nm, 500 to 640 nm, or 500 to 650 nm, and an emission wavelength ranging from 550 to 570 nm, 550 to 590 nm, 550 to 605 nm, 550 to 615 nm, 550 to 625 nm, 550 to 640 nm, or 550 to 650 nm. Each possibility represents a separate embodiment of the invention.

In some the protein product of the herein disclosed at least one reporter gene has an excitation wavelength ranging from 500 to 650 nm an emission wavelength ranging from 550 to 650 nm.

It should be appreciated that in some embodiments, a reporter gene as used herein comprises a plurality of reporter genes. In some embodiments, each reporter gene of a plurality of reporter genes may be integrated into at least one position or location in gender chromosome Z. In some embodiments, a plurality is at least 2, at least 5, at least 8, at least 10, or any value and range therebetween. Each possibility represents a separate embodiment of the invention. In some embodiments, a plurality is 2-6, 3-10, 4-8, or 2-12. Each possibility represents a separate embodiment of the invention. In some embodiments, a plurality of reporter genes comprises a plurality of different reporter genes. In some embodiments, a plurality of reporter genes comprises a plurality of the same reporter gene (e.g., multiple copies). A non-limiting example of a plurality of different reporter genes includes, but is not limited to, a transgenic subject comprising a fluorescent protein (e.g., green or red fluorescence protein) and a bioluminescent protein (e.g., luciferase). A non-limiting example of a plurality of the same reporter gene includes, but is not limited to multiple copies of the same reporter gene, e.g., an exogenous sequence comprising two or more sequences of a fluorescent protein (e.g., green or red fluorescence protein) or a bioluminescent protein (e.g., luciferase).

In some embodiments, when a plurality of reporter genes are provided, e.g., by crossing a female transgenic avian subject comprising at least one first reporter gene integrated into the gender chromosome W with a male transgenic avian subject comprising at least one second reporter gene integrated into both gender chromosomes Z, the detecting step comprises applying an excitation wavelength ranging from 500 to 650 nm to at least one egg obtained by the crossing, and determining an emission wavelength ranging from 550 to 650 nm. In some embodiments, upon excitation at wavelengths as disclosed herein, a male embryo emits a wavelength derived from the protein product of the at least one first reporter gene or the at least one second reporter gene. In some embodiments, upon excitation at wavelengths as disclosed herein, a female embryo emits a wavelength derived from the combination of the protein products of the at least one first reporter gene and the at least one second reporter gene.

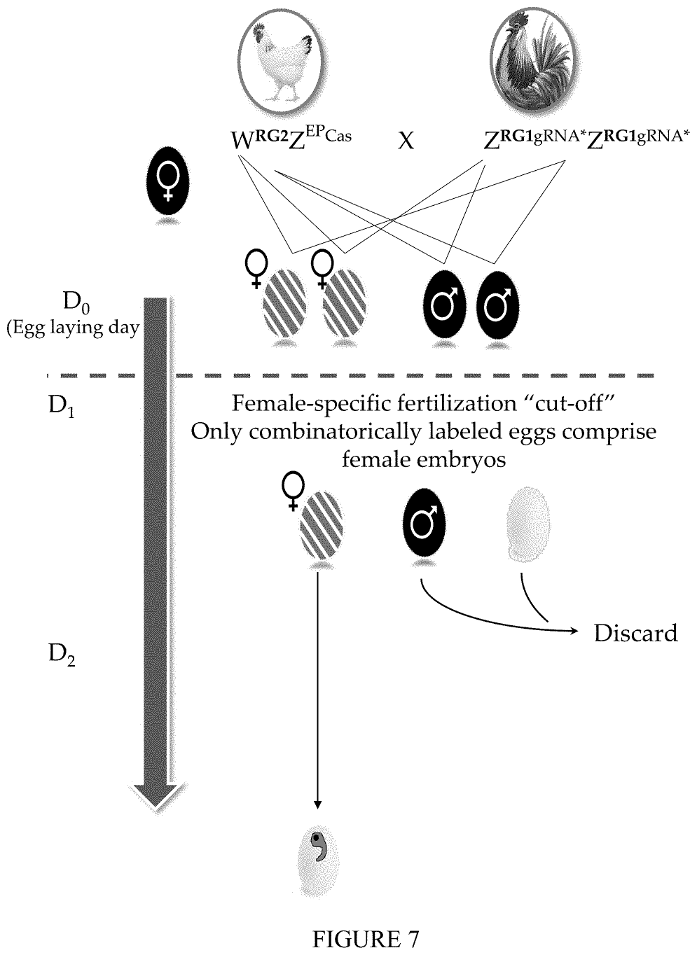

As a non-limiting example (as depicted in FIG. 7), crossing a female transgenic avian subject comprising a first reporter gene encoding a GFP integrated into the gender chromosome W, with a male transgenic avian subject comprising a second reporter gene encoding RFP integrated into both gender chromosomes Z, the detecting step comprises applying an excitation wavelength ranging from 500 to 650 nm to at least one egg obtained by the crossing, and determining an emission wavelength ranging from 550 to 650 nm. According to this non-limiting example, upon excitation at wavelengths as disclosed herein, a male embryo emits a wavelength derived solely from the RFP protein product (“RFP signal”), whereas a female embryo emits a wavelength derived from the combination of RFP and GFP (“RFP derivative signal”).

In some embodiments, the method of the invention is directed to determining an unhatched fertilized egg comprises a male embryo, wherein a RFP signal is detected in the unhatched egg upon excitation as disclosed herein.

In some embodiments, the method of the invention is directed to determining an unhatched fertilized egg comprises a female embryo, wherein a RFP-derivative signal is detected in the unhatched egg upon excitation as disclosed herein.

As used herein, the term “RFP derivative signal” refers to any fluorescently emitted light provided by exciting RFP and at least one more fluorescent label, wherein the excitation is in a wavelength range ranging from 500 nm to 650 nm, wherein the emission is in a wavelength ranging from 550 nm to 650 nm, and wherein the emission of the combination of RFP and at least one more fluorescent protein is distinguishable from the emission of RFP alone.

In some embodiments, the determination of the detectable signal formed, is indicative of the gender of the examined embryo.

As used herein, the term “fluorescence” refers to the emission of light by a substance that has been illuminated, and absorbed light or other electromagnetic radiation. In some embodiments, fluorescence is a form of luminescence.

As used herein, the term “luminesce” refers to an emission of light by a substance not resulting from heat. In some embodiments, luminescence comprises bioluminescence. As used herein, the term “bioluminescence” refers to luminescence that is a result of a biochemical reactions in vivo (e.g., in an organism), or in vitro.

In some embodiments, the at least one reporter gene encodes a red fluorescent protein (RFP).

As used herein, the term “red fluorescent protein” or “RFP” refers to a fluorescent protein that emits orange, red, and far-red fluorescence that has been isolated from anthozoans (corals) and anemones as well as any variants thereof. There are two main types of RFP proteins, DsRed and Kaede. DsRed-like RFPs are derived from Discosoma striata and include but are not limited to mCherry, zFP538, mKO, mOrange, mRouge, E2-Crimson, mNeptune, TagRFP657, Keima, mKate, mStrawberry, mBanana, mHoneydew, niTangerine, mRaspberry, mPlum, mRFPmars and mRFPruby.

In some embodiments, a fluorescent reporter gene comprises a green fluorescence protein (GFP) that is not the wild type GFP. In some embodiments, the GFP is a mutant GFP. In some embodiments, the mutant GFP is characterized by a red-emitted fluorescence. In some embodiments, red-emitted fluorescence GFP which can be utilized according to the herein disclosed method is characterized by an excitation wavelength and an emission wavelength as mentioned above for RFP. In some embodiments, a red-emitted fluorescence GFP which can be utilized according to the herein disclosed method is characterized by an excitation wavelength maxima at 555 nm, and an emission wavelength maxima at 585 nm.

Specific mutants of GFP characterized by red-emitted fluorescence are common and would be apparent to one of ordinary skill in the art. Non-limiting examples of such mutants include, but are not limited to, any one of the following mutations: Serine 65, Asparagine 68, F46L, V163A, I167V, and any combination thereof.

In another embodiment, the fluorescent reporter gene is selected from: red fluorescent protein (RFP), cyan fluorescent protein (CFP), yellow fluorescent protein (YFP), and auto-fluorescent proteins including blue fluorescent protein (BFP).

In another embodiment, the reporter gene comprises a YFP. In some embodiments, the YFP is selected from: enhanced YFP (e.g., eYFP), Citrine YFP, Venus YFP, and ZsYellow1 YFP.

In another embodiment, the reporter gene a CFP. In some embodiments, the CFP is AmCyan1.

It should be understood that in yet some further embodiments, the reporter gene that may be applicable in the methods, transgenic avian subjects, constructs, cells and kits of the invention may be any of the fluorescent proteins disclosed in Table 2 herein after.

TABLE 2
fluorescent proteins:
Orange Fluorescent Proteins

| | | |
|---|---|---|
| Kusabira Orange | 548 | 559 |
| Kusabira Orange2 | 551 | 565 |
| mOrange | 548 | 562 |
| mOrange2 | 549 | 565 |
| dTomato | 554 | 581 |
| dTomato-Tandem | 554 | 581 |
| TagRFP | 555 | 584 |
| TagRFP-T | 555 | 584 |
| DsRed | 558 | 583 |
| DsRed2 | 563 | 582 |
| DsRed-Express (T1) | 555 | 584 |
| DsRed-Monomer | 556 | 586 |
| mTangerine | 568 | 585 |

Yellow Fluorescent Proteins

| | | |
|---|---|---|
| EYFP | 514 | 527 |
| Topaz | 514 | 527 |
| Venus | 515 | 528 |
| mCitrine | 516 | 529 |
| YPet | 517 | 530 |
| TagYFP | 508 | 524 |
| PhiYFP | 525 | 537 |
| ZsYellow1 | 529 | 539 |
| mBanana | 540 | 553 |

Red Fluorescent Proteins

| | | |
|---|---|---|
| mRuby | 558 | 605 |
| mApple | 568 | 592 |
| mStrawberry | 574 | 596 |
| AsRed2 | 576 | 592 |
| mRFP1 | 584 | 607 |
| JRed | 584 | 610 |
| mCherry | 587 | 610 |
| HcRed1 | 588 | 618 |
| mRaspberry | 598 | 625 |
| dKeima-Tandem | 440 | 620 |
| HcRed-Tandem | 590 | 637 |
| mPlum | 590 | 649 |
| AQ143 | 595 | 655 |

In some embodiments, the reporter gene within the transgenic avian of the invention may be at least one bioluminescent reporter gene.

In some embodiments, the bioluminescent reporter gene is luciferase. The term “Luciferase” refers hereinafter to a class of oxidative enzymes that produce bioluminescence (photon emission). The emitted photon can be detected by light sensitive apparatus such as a luminometer or modified optical microscopes.

In some embodiments, the reporter gene being a luciferase catalyzes a reaction characterized by the production of visible light having a wavelength ranging from 550-600 nm. In some embodiments, the produced light has a wavelength ranging from 550-575 nm, or 550-595 nm. In some embodiments, the method further comprises a step of supplementing an exogenous substrate (e.g., luciferin) such as for enabling the light reaction to occur.

In some embodiments, the present method further comprises a step of detecting at least one detectable signal of the protein product in the fertilized unhatched egg, wherein detection of the at least one detectable signal is indicative of the expression of the reporter gene in the fertilized egg, thereby determining that the fertilized unhatched egg comprises a male embryo.

In some embodiments, the method comprises determining a detectable signal, wherein the signal correlates with the presence and/or expression of the at least one reporter gene and/or a product thereof, and thereby is indicative of the presence of a specific gender chromosome in the examined fertilized unhatched egg. In some embodiments, the detectable signal is a change in that is perceptible by observation, either by using instruments or not. In some embodiments, the signal is detected directly or indirectly. In some embodiments, the signal is an optical signal.

In some embodiments, the step of detecting the at least one detectable signal is performed 1 hr at most post egg laying, 2 hr at most post egg laying, 3 hr at most post egg laying, 4 hr at most post egg laying, 6 hr at most post egg laying, 8 hr at most post egg laying, 10 hr at most post egg laying, 12 hr at most post egg laying, 16 hr at most post egg laying, 18 hr at most post egg laying, 22 hr at most post egg laying, 24 hr at most post egg laying, 1 day at most post egg laying, 2 days at most post egg laying, or 3 days at most post egg laying, or any value and range therebetween. Each possibility represents a separate embodiment of the invention. In some embodiments, the step of detecting the at least one detectable signal is performed 1 hr to 72 hr at most post egg laying, 2 hr to 48 hr at most post egg laying, 3 hr to 60 hr at most post egg laying, 6 hr to 72 hr at most post egg laying, 1 hr to 24 hr at most post egg laying, 4 hr to 54 hr at most post egg laying, 7 hr to 45 hr at most post egg laying, 10 hr to 70 hr at most post egg laying, 12 hr to 52 hr at most post egg laying, or 18 hr to 66 hr at most post egg laying. Each possibility represents a separate embodiment of the invention.

In some embodiments, detecting comprises subjecting the fertilized unhatched egg to a light source.

In some embodiments, a light source comprises a wavelength ranging from 400 nm to 650 nm. In some embodiments, light of any wavelength may be used with the proviso that the light source is not in the ultra-violet (UV) light range, e.g., 10-400 nm.

In some embodiments, the step of subjecting the egg to a light source provides excitation of at least one fluorescent reporter protein. In some embodiments, the excitation wavelength is between 500 nm to 650 nm. In some embodiments, the excitation wavelength is at least 510 nm, at least 515 nm, at least 520 nm, at least 525 nm, at least 530 nm, at least 535 nm, at least 540 nm, at least 545 nm, at least 550 nm, at least 555 nm, at least 560 nm, at least 565 nm, at least 570 nm, at least 575 nm, at least 580 nm, at least 585 nm, at least 590 nm, at least 595 nm, at least 600 nm, at least 605 nm, at least 610 nm, at least 615 nm, at least 620 nm, at least 625 nm, at least 630 nm, at least 635 nm, at least 640 nm, or any value and range therebetween. Each possibility represents a separate embodiment of the invention.

In one embodiment, the light source may be provided by a laser.

In some embodiments, the fertilized unhatched egg is exposed to a light source. In some embodiments, the fertilized unhatched egg is placed in a position facilitating exposure of the embryo at any stage to the light source. In some embodiments, a region containing the upper face of the egg yolk at any stage of the embryo is illuminated or excited with the light source.

For example, a step of subjecting the fertilized unhatched egg to a light source can be performed using a system, apparatus or a device that may comprise a laser source, a stand for the fertilized unhatched egg, a lens, a filter, a stand for a detector and a detector.

As used herein, the term “detector” refers to any type of device that detects and/or measures light.

In some embodiments, it should be noted that a detectable signal, for example a fluorescent signal is detected using suitable fluorescent means. In some embodiments, the detectable signal formed by the fluorescent reporter gene or a combination thereof, for example RFP, YFP, GFP, or any combination thereof, may be detected by light sensitive apparatus such as modified optical microscopes or charge coupled device (CCD), a highly sensitive photon detector.

In some embodiments, the methods of the invention comprises using any device, apparatus or system that provides at least one light source, at least one detector, at least one filter, and at least one holding arm that places the egg in an appropriate position facilitating the exposure of cells expressing the reporter gene of the invention to the light source.

In some embodiments, any one of the first nucleic acid sequence and the second nucleic acid sequence encodes at least one guide RNA (gRNA) or at least one CRISPR associated protein 9 (Cas9) protein.

In some embodiments, any one of the first nucleic acid sequence and the second nucleic acid sequence is integrated into the gender chromosome Z of any one of the female transgenic avian subject and the male avian subject. In some embodiments, integrating comprises contacting or co-transfecting at least one cell of any one of the female transgenic avian subject and the male transgenic avian subject, with: at least one Cas protein encoding sequence or at least one nucleic acid sequence encoding thereof, and at least one gRNA or at least one nucleic acid sequence encoding thereof, wherein the gRNA targets at least one protospacer within the at least one gender chromosome Z.

In some embodiments, integrating comprises contacting or co-transfecting at least one cell of the male transgenic avian subject, with: at least one Cas protein encoding sequence or at least one nucleic acid sequence encoding thereof, and at least one gRNA or at least one nucleic acid sequence encoding thereof, wherein the gRNA targets at least one protospacer within both gender chromosomes Z of the male avian subject.

As indicated herein before, the method of the invention involves the provision of at least one transgenic avian subject. The preparation of a transgenic avian subject requires the use of genetic engineering approach that may use specific gene editing compound or component. Non-limiting examples for gene editing components include nucleases.

In some embodiments, the present invention is directed to a kit comprising the herein disclosed nucleic acids and/or exogenous genes. In some embodiments, the kit is for preparing the herein disclosed at least one transgenic avian subject.

In some embodiments, the kit comprises: (a) at least one first nucleic acid sequence encoding any one of at least one Cas protein and at least one gRNA; (b) a second nucleic acid sequence, wherein when the first nucleic acid sequence encodes at least one Cas protein the second nucleic acid encodes at least one gRNA, wherein when the first nucleic acid sequence encodes at least one gRNA said second nucleic acid encodes at least one Cas protein, and wherein the combination of the at least one Cas protein and the at least one gRNA targets at least one vital avian gene.

In some embodiments, the kit optionally comprises a third nucleic acid, a fourth nucleic acid sequence, or both, encoding at least one reporter gene having an excitation wavelength ranging from 500 to 650 nm and an emission wavelength ranging from 550 to 650 nm nucleic acid sequence.

In some embodiments, the kit comprises instruction for the preparation of: (i) at least one transgenic male avian subject comprising the first nucleic acid sequence integrated into at least one locus within both gender chromosomes Z of the male avian subject, (ii) at least one transgenic female avian subject comprising the second nucleic acid sequence and optionally the third nucleic acid, wherein each of the second nucleic acid and the third nucleic acid is integrated into at least one locus within gender chromosome Z of the female avian subject, or (i) and (ii).

In some embodiments, a transgenic male avian subject prepared according to the herein disclosed method or kit, is provided. In some embodiments, a transgenic male avian subject comprising at least one first nucleic acid sequence encoding any one of at least one Cas protein and at least one gRNA integrated into at least one locus within both gender chromosomes Z, is provided.

In some embodiments, a transgenic female avian subject prepared according to the herein disclosed method or kit, is provided. In some embodiments, a transgenic female avian subject comprising a second nucleic acid sequence encoding any one of at least one Cas protein and at least one gRNA integrated into at least one locus within gender chromosome Z, is provided. In some embodiments, the herein provided transgenic female avian subject, further comprises the herein disclosed third nucleic acid sequence.

In some embodiments, the further comprises instructions for breeding the transgenic male avian subject with the transgenic female avian subject so as to obtain a progeny.

In some embodiments, there is provided a progeny resulting from the breeding of the transgenic male avian subject of the invention with the transgenic female avian subject of the invention.

In some embodiments, there is provided a female progeny or offspring of the transgenic male avian subject of the invention with the transgenic female avian subject of the invention.

In some embodiments, there is provided a male-free flock of the transgenic male avian subject of the invention with the transgenic female avian subject of the invention.

In some embodiments, genetic engineering comprises integration of an exogenous nucleic acid into the genome of a cell or an organism comprising same. In some embodiments, the method of the invention comprises a step of integrating any one of the first nucleic acid sequence, the second nucleic acid sequence, the third nucleic acid sequence, and any combination thereof, into a chromosome of the at least one transgenic avian subject of the invention. In some embodiments, the method comprises integrating any one of the first nucleic acid sequence and the second nucleic acid sequence into at least one site in locus 42172748-42177748 of the gender chromosome Z. In some embodiments, the method further comprises integrating the third nucleic acid sequence into at least one site in locus 42172748-42177748 of the gender chromosome Z.

In some specific embodiments, a nuclease applicable according to the herein discloses method comprises a RNA-guided DNA binding protein nuclease.

As used herein, the term “RNA-guided DNA binding protein nuclease” refers to any nuclease which is guided to its cleavage site (or alternatively, a site for any other alternative activity), by a RNA molecule. In some embodiments, the RNA molecule is a guide RNA molecule.

In some embodiments, the exogenous gene is integrated into the gender or sex chromosome of the transgenic avian subject or animal provided by the method of the invention using at least one programmable engineered nuclease (PEN). The term “programmable engineered nucleases (PEN)” as used herein, refers to synthetic enzymes that cut specific DNA sequences, derived from natural occurring nucleases involved in DNA repair of double strand DNA lesions and enabling direct genome editing.

In some embodiments, PEN used by the methods of the invention may be any one of a clustered regularly interspaced short palindromic repeat (CRISPR) Class 2 or Class 1 system.

The clustered regularly interspaced short palindromic repeats (CRISPR) Type II system is a bacterial immune system that has been modified for genome engineering. It should be appreciated however that other genome engineering approaches, like zinc finger nucleases (ZFNs) or transcription-activator-like effector nucleases (TALENs) that relay upon the use of customizable DNA-binding protein nucleases that require design and generation of specific nuclease-pair for every genomic target may be also applicable herein.

CRISPR-Cas systems fall into two classes. Class 1 systems use a complex of multiple Cas proteins to degrade foreign nucleic acids. Class 2 systems use a single large Cas protein for the same purpose. More specifically, Class 1 may be divided into types I, III, and IV and class 2 may be divided into types II, V, and VI.

As used herein, “CRISPR arrays” also known as SPIDRs (Spacer Interspersed Direct Repeats) constitute a family of recently described DNA loci that are usually specific to a particular bacterial species. The CRISPR array is a distinct class of interspersed short sequence repeats (SSRs) that were first recognized in E. coli. In subsequent years, similar CRISPR arrays were found in Mycobacterium tuberculosis, Haloferax mediterranei, Methanocaldococcus jannaschii, Thermotoga maritima and other bacteria and archaea. It should be understood that the invention contemplates the use of any of the known CRISPR systems, particularly and of the CRISPR systems disclosed herein. The CRISPR-Cas system has evolved in prokaryotes to protect against phage attack and undesired plasmid replication by targeting foreign DNA or RNA. The CRISPR-Cas system, targets DNA molecules based on short homologous DNA sequences, called spacers that exist between repeats. These spacers guide CRISPR-associated (Cas) proteins to matching (and/or complementary) sequences within the foreign DNA, called proto-spacers, which are subsequently cleaved. The spacers can be rationally designed to target any DNA sequence. Moreover, this recognition element may be designed separately to recognize and target any desired target. With respect to CRISPR systems, as will be recognized by those skilled in the art, the structure of a naturally occurring CRISPR locus includes a number of short repeating sequences generally referred to as “repeats”. The repeats occur in clusters and are usually regularly spaced by unique intervening sequences referred to as “spacers.” Typically, CRISPR repeats vary from about 24 to 47 base pair (bp) in length and are partially palindromic. The spacers are located between two repeats and typically each spacer has unique sequences that are from about 20 or less to 72 or more bp in length. In some embodiments the CRISPR spacers used in the sequence encoding at least one gRNA of the methods and kits of the invention comprise between 10 to 75 nucleotides (nt) each. In some embodiments, the gRNA comprises at least: 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, or any vale and range therebetween. Each possibility represents a separate embodiment of the invention. In some embodiments, the gRNA comprises 70 to 150 nt. In some specific embodiments the spacers comprise 20 to 35 nucleotides.

In addition to at least one repeat and at least one spacer, a CRISPR locus also includes a leader sequence and optionally, a sequence encoding at least one tracrRNA. The leader sequence typically is an AT-rich sequence of up to 550 bp directly adjoining the 5′ end of the first repeat.

In some embodiments, the PEN used by the methods of the invention may be a CRISPR Class 2 system. In yet some further particular embodiments, such class 2 system may be a CRISPR type II system.

More specifically, three major types of CRISPR-Cas system are delineated: Type I, Type II and Type III.

The type II CRISPR-Cas systems include the ‘HNH’-type system (Streptococcus-like; also known as the Nmeni subtype, for Neisseria meningitidis serogroup A str. Z2491, or CASS4), in which Cas9, a single, very large protein, seems to be sufficient for generating crRNA and cleaving the target DNA, in addition to the ubiquitous Cas 1 and Cas2. Cas9 contains at least two nuclease domains, a RuvC-like nuclease domain near the amino terminus and the HNH (or McrA-like) nuclease domain in the middle of the protein, but the function of these domains remains to be elucidated. However, as the HNH nuclease domain is abundant in restriction enzymes and possesses endonuclease activity responsible for target cleavage.

Type II systems cleave the pre-crRNA through an unusual mechanism that involves duplex formation between a tracrRNA and part of the repeat in the pre-crRNA; the first cleavage in the pre-crRNA processing pathway subsequently occurs in this repeat region. Still further, it should be noted that type II system comprise at least one of Cas9, Cas1, Cas2 csn2, and Cas4 genes. It should be appreciated that any type II CRISPR-Cas systems may be applicable in the present invention, specifically, any one of type II-A or B.

In some embodiments, the at least one Cas gene used in the methods and kits of the invention may be at least one Cas gene of type II CRISPR system (either typeII-A or typeII-B). In some embodiments, at least one Cas gene of type II CRISPR system used by the methods and kits of the invention is the Cas9 gene. It should be appreciated that such system may further comprise at least one of Cas1, Cas2, csn2 and Cas4 genes.

In some embodiments, a Cas protein consists or comprise a Cas9 protein.

Double-stranded DNA (dsDNA) cleavage by Cas9 is a hallmark of “type II CRISPR-Gas” immune systems. The CRISPR-associated protein Cas9 is an RNA-guided DNA endonuclease that uses RNA:DNA complementarity to identify target sites for sequence-specific double stranded DNA (dsDNA) cleavage, creating the double strand brakes (DSBs) required for the HDR that results in the integration of the reporter gene into the specific target sequence, for example, a specific target within the avian gender chromosome Z. The targeted DNA sequences are specified by the CRISPR array, which is a series of about 30 to 40 bp spacers separated by short palindromic repeats. The array is transcribed as a pre-crRNA and is processed into shorter crRNAs that associate with the Cas protein complex to target complementary DNA sequences known as proto-spacers. These proto-spacer targets must also have an additional neighboring sequence known as a proto-spacer adjacent motif (PAM) that is required for target recognition. After binding, a Cas protein complex serves as a DNA endonuclease to cut both strands at the target and subsequent DNA degradation occurs via exonuclease activity.

CRISPR type II system as used herein requires the inclusion of two essential components: a “guide” RNA (gRNA) and a non-specific CRISPR-associated endonuclease (Cas9). The gRNA is a short synthetic RNA composed of a “scaffold” sequence necessary for Cas9-binding and about 20 nucleotide long “spacer” or “targeting” sequence which defines the genomic target to be modified. Thus, one can change the genomic target of Cas9 by simply changing the targeting sequence present in the gRNA. Guide RNA (gRNA), as used herein refers to a synthetic fusion of the endogenous bacterial crRNA and tracrRNA, providing both targeting specificity and scaffolding/binding ability for Cas9 nuclease. Also referred to as “single guide RNA” or “sgRNA”. CRISPR was originally employed to “knock-out” target genes in various cell types and organisms, but modifications to the Cas9 enzyme have extended the application of CRISPR to “knock-in” target genes, selectively activate or repress target genes, purify specific regions of DNA, and even image DNA in live cells using fluorescence microscopy. Furthermore, the ease of generating gRNAs makes CRISPR one of the most scalable genome editing technologies and has been recently utilized for genome-wide screens.

The region within the genome to be edited, specifically, the specific target loci within the gender or sex chromosome Z, W, or both, wherein any one of the first nucleic acid sequence (e.g., encoding a gRNA or Cas9 protein), the second nucleic acid sequence (e.g., encoding a Cas9 protein or a gRNA), the third nucleic acid sequence (e.g., encoding at least one first reporter gene), and the fourth nucleic acid sequence (e.g., encoding at least one second reporter gene), are to be integrated, should be present upstream of a protospacer adjacent motif (PAM).

The PAM sequence is absolutely necessary for target binding and the exact sequence is dependent upon the species of Cas9 (5′ NGG 3′ for Streptococcus pyogenes Cas9). In some embodiments, Cas9 from S. pyogenes is used in the methods and kits of the invention. Nevertheless, it should be appreciated that any known Cas9 may be applicable. Non-limiting examples for Cas9 useful in the present disclosure include but are not limited to Streptococcus pyogenes (SP), also indicated herein as SpCas9, Staphylococcus aureus (SA), also indicated herein as SaCas9, Neisseria meningitidis (NM), also indicated herein as NmCas9, Streptococcus thermophilus (ST), also indicated herein as StCas9 and Treponema denticola (TD), also indicated herein as TdCas9. In some specific embodiments, the Cas9 of Streptococcus pyogenes M1 GAS, specifically, the Cas9 of protein id: AAK33936.1, may be applicable in the methods and kits of the invention. Once expressed, the Cas9 protein and the gRNA, form a riboprotein complex through interactions between the gRNA “scaffold” domain and surface-exposed positively-charged grooves on Cas9. Cas9 undergoes a conformational change upon gRNA binding that shifts the molecule from an inactive, non-DNA binding conformation, into an active DNA-binding conformation. Importantly, the “spacer” sequence of the gRNA remains free to interact with target DNA. The Cas9-gRNA complex binds any genomic sequence with a PAM, but the extent to which the gRNA spacer matches the target DNA determines whether Cas9 will cut. Once the Cas9-gRNA complex binds a putative DNA target, a “seed” sequence at the 3′ end of the gRNA targeting sequence begins to anneal to the target DNA. If the seed and target DNA sequences match, the gRNA continues to anneal to the target DNA in a 3′ to 5′ direction.

Cas9 will only cleave the target if sufficient homology exists between the gRNA spacer and target sequences. Still further, the Cas9 nuclease has two functional endonuclease domains: RuvC and HNH. Cas9 undergoes a second conformational change upon target binding that positions the nuclease domains to cleave opposite strands of the target DNA. The end result of Cas9-mediated DNA cleavage is a double strand break (DSB) within the target DNA that occurs about 3 to 4 nucleotides upstream of the PAM sequence.

The resulting DSB may be then repaired by one of two general repair pathways, the efficient but error-prone non-homologous end joining (NHEJ) pathway and the less efficient but high-fidelity homology directed repair (HDR) pathway. In some embodiments, the insertion that results in the specific integration of any one of the first nucleic acid sequence, the second nucleic acid sequence, the third nucleic acid sequence, and the fourth nucleic acid sequence of the invention to the at least one specific target locus within the at least one gender chromosome Z, both gender chromosomes Z, gender chromosome W, or any combination thereof, is a result of repair of DSBs caused by a Cas protein, e.g., Cas9. In some embodiments, any one of the first nucleic acid sequence, the second nucleic acid sequence, and the fourth nucleic acid sequence of the invention is integrated, or knocked-in the target locus by HDR. In some embodiments, an organism, e.g., an embryo, comprising the first and second nucleic acids of the invention is obtained from breeding or crossing at least one transgenic female avian of the invention with at least one transgenic male of the invention. In some embodiments, the organism, e.g., embryo is expressing both the first and the second nucleic acids of the invention. In some embodiments, expression of both the first and the second nucleic acids of the invention provides a gRNA-Cas complex. In some embodiments, the gRNA-Cas complex knocks out a target gene. In some embodiments, the knocked out target gene is a vital gene as disclosed herein. In some embodiments, the gRNA-Cas complex knocks in a target gene. In some embodiments, the knocked in target gene is the at least one reporter gene as disclosed herein.

In some embodiments, the first or second nucleic acid sequence of the invention encode at least two gRNA. In some embodiments, the at least two gRNA complex with a Cas protein. In some embodiments, the at least two gRNA comprise different nucleic acid sequence. In some embodiments, a first gRNA comprises a nucleic acid sequence capable of guiding the Cas protein to knock out a vital gene as disclosed herein and a second gRNA comprises a nucleic acid sequence capable of guiding the Cas protein to knock in the at least one reporter gene disclosed herein, and vice versa.

In some further embodiments, the gRNA of the kit of the invention may comprise at least one CRISPR RNA (crRNA) and at least one trans-activating crRNA (tracrRNA).

As indicated herein, the gRNA of the kit of the invention may be complementary, at least in part, to a target genomic DNA.

In nature complementarity is the base principle of DNA replication and transcription as it is a property shared between two DNA or RNA sequences, such that when they are aligned antiparallel to each other, the nucleotide bases at each position in the sequences will be complementary (e.g., A and T or U, C and G).

Thus, for the preparation of a transgenic avian animal used by the methods of the invention, as well as the kits described herein after, at least two nucleic acid sequences or molecules are provided.

Still further, a “gRNA” or “targeting RNA” is a RNA that, when transcribed from the portion of the CRISPR system encoding it, comprises at least one portion of RNA sequence that is identical to (with the exception of replacing T for U in the case of RNA) or complementary to (and thus “targets”) a DNA sequence in the target genomic DNA, referred to herein as a protospacer. The CRISPR systems of the present disclosure may optionally encode more than one targeting RNA, and the targeting RNAs be directed to one or more target sequences in the genomic DNA. A “proto-spacer”, as used herein, refers to the target sequence within the target chromosome. Such proto-spacers comprise nucleic acid sequence having sufficient complementarity to a targeting RNA encoded by the CRISPR spacers comprised within the nucleic acid sequence encoding the gRNA of the methods and kits of the invention.

As used herein, “nucleic acids or nucleic acid molecules” is interchangeable with the term “polynucleotide(s)” and it generally refers to any polyribonucleotide or poly-deoxyribonucleotide, which may be unmodified RNA or DNA or modified RNA or DNA or any combination thereof. “Nucleic acids” include, without limitation, single-and double-stranded nucleic acids. As used herein, the term “nucleic acid(s)” also includes DNAs or RNAs as described above that contain one or more modified bases. As used herein, the term “oligonucleotide” is defined as a molecule comprised of two or more deoxyribonucleotides and/or ribonucleotides, and preferably more than three. Its exact size will depend upon many factors which in turn, depend upon the ultimate function and use of the oligonucleotide. The oligonucleotides may be from 8 to 1,000 nucleotides long. More specifically, the oligonucleotide molecule/s used by the kit of the invention comprises at least: 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100, 200, 300, 400, 500, 600, 700, 800, 900, 1000 bases in length, or any value and range therebetween. Each possibility represents a separate embodiment of the invention. In another embodiment, the oligonucleotide molecule/s comprises 1,000 to 3,000 bases in length.

Nucleic acid molecules can be composed of monomers that are naturally-occurring nucleotides (such as DNA and RNA), or analogs of naturally-occurring nucleotides (e.g., alpha-enantiomeric forms of naturally-occurring nucleotides), or modified nucleotides or any combination thereof. Herein this term also encompasses a cDNA, i.e. complementary or copy DNA produced from an RNA template by the action of reverse transcriptase (RNA-dependent DNA polymerase).

In this connection an “isolated polynucleotide” is a nucleic acid molecule that is separated from the genome of an organism. For example, a DNA molecule that encodes a reporter gene used by the methods and kits of the invention or any derivatives or homologs thereof, as well as the sequences encoding the CRISPR/Cas9 and gRNAs of the methods and kits of the invention, that has been separated from the genomic DNA of a cell is an isolated DNA molecule. Another example of an isolated nucleic acid molecule is a chemically-synthesized nucleic acid molecule that is not integrated in the genome of an organism. A nucleic acid molecule that has been isolated from a particular species is smaller than the complete DNA molecule of a chromosome from that species.

In some embodiments, the nucleic acid sequences used by the methods and kits (described herein after) of the invention, specifically, nucleic acid sequences comprising sequences encoding the Cas and gRNA, or alternatively the at least one reporter gene, may be provided constructed within a vector, cassette or any other vehicle. The invention thus further relates to recombinant DNA constructs comprising the polynucleotides of the invention, and optionally, further additional elements such as promoters, regulatory and control elements, translation, expression and other signals, operably linked to the nucleic acid sequence of the invention. A non-limiting example for a vector provided by the invention may be any vector that comprises the nucleic acid sequences that encode the Cas9 and specific gRNA required for integration of the reporter gene into the target gender chromosome.

Still further, the invention further encompasses any host cell that comprises and/or expresses DNA constructs comprising any of the nucleic acid sequences of the invention, and/or vectors comprising same.

As used herein, the terms “recombinant DNA”, “recombinant nucleic acid sequence” or “recombinant gene” refer to a nucleic acid comprising an open reading frame encoding one of the CRISPR system components of the invention, specifically, the CRISPR/Cas9 type II, along with the gRNA of the invention that targets the Cas to the corresponding protospacer in a specific locus within the avian gender chromosome Z. In some embodiments, recombinant DNA as used herein further refers to a nucleic acid sequence comprising an open reading frame encoding an exogenous gene of the invention, such as the at least one reporter gene.

It should be appreciated that in some embodiments, at least one of the first and the second nucleic acid sequences provided and used by the methods and kits of the invention may be constructed and comprised within a vector. “Vectors” or “Vehicles”, as used herein, encompass vectors such as plasmids, phagemides, viruses, integratable DNA fragments, and other vehicles, which enable the integration of DNA fragments into the genome of the host, or alternatively, enable expression of genetic elements that are not integrated. Vectors are typically self-replicating DNA or RNA constructs containing the desired nucleic acid sequences, and operably linked genetic control elements that are recognized in a suitable host cell and effect the translation of the desired spacers. Generally, the genetic control elements can include a prokaryotic promoter system or a eukaryotic promoter expression control system. Such system typically includes a transcriptional promoter, transcription enhancers to elevate the level of RNA expression. Vectors usually contain an origin of replication that allows the vector to replicate independently of the host cell. In some embodiments, the expression vectors used by the invention may comprise elements necessary for integration of sequence cloned within the vector, e.g., the first nucleic acid sequence, the second nucleic acid sequence, the third nucleic acid sequence comprising at least one reporter gene, or any combination thereof, into the avian gender chromosome Z.

Accordingly, the term “a control and/or regulatory element” includes a promoter, a terminator, and other expression control elements. Such regulatory elements are described in Goeddel; [Goeddel., et al., Gene Expression Technology: Methods in Enzymology 185, Academic Press, San Diego, Calif. (1990)]. For instance, any of a wide variety of expression control sequences that control the expression of a DNA sequence when operably linked to it may be used in these vectors to express DNA sequences encoding any desired protein using the method of this invention.

A vector may additionally include appropriate restriction sites, antibiotic resistance or other markers for selection of vector-containing cells. Plasmids are the most commonly used form of vector but other forms of vectors which serve an equivalent function, and which are, or become, known in the art are suitable for use herein. See, e.g., Pouwels et al., Cloning Vectors: a Laboratory Manual (1985 and supplements), Elsevier, N.Y.; and Rodriquez, et al. (eds.) Vectors: a Survey of Molecular Cloning Vectors and their Uses, Buttersworth, Boston, Mass (1988), which are incorporated herein by reference.

In some embodiments, preparation of a transgenic avian subject used by the methods of the invention, requires the preparation of an avian cell comprising at least one reporter gene integrated into specific loci within the at least one gender chromosome Z or both, or gender chromosome W, or a combination thereof. Such cell may be prepared, in some embodiments, by contacting, introducing or co-transfecting the cell of the avian subject or any cell introduced to the subject, with the first and the second nucleic acid sequences provided by the methods and kits of the invention or with any construct, vector, vehicle, comprising same. Such cell may be prepared, in some embodiments, by contacting, introducing or co-transfecting the cell of the avian subject or any cell introduced to the subject, with first, the second, the third, the fourth nucleic acid sequences, or any combination thereof, provided by the methods and kits of the invention or with any construct, vector, vehicle, comprising same. In some embodiments, the first and the second nucleic acid sequences encode the gene editing elements, and the third nucleic acid sequence encodes the at least one reporter gene, specifically, RFP.

In some embodiments, the first and the second nucleic acid sequences encode the gene editing elements, and the third nucleic acid sequence and the fourth nucleic acid sequence encode RFP and at least another one reporter gene, specifically, a fluorescent protein having suitable for excitation in a wavelength as disclosed herein so as to emit a detectable signal in an emission wavelengths as disclosed herein.

The insertion and specific integration of the at least one reporter gene to the specific target locus within a gender chromosome Z or W of the transgenic avian subject, involves, in some embodiments, the provision of the CRISPR/Cas system that includes specific gRNA and the nucleic acid sequence of the at least one reporter gene that should be integrated. To facilitate and enable integration, the at least one reporter gene is flanked, in some embodiments, with sequences that may be homolog to the sequences flanking the targeted integration site and thereby enable recombination. In some embodiments, the at least one reporter gene of the third nucleic acid sequence is flanked at 5′ and 3′ thereof by homologous arms. In some embodiments, these arms are required to facilitate HDR of the at least one reporter gene at the integration site.

The removal or excision of the vital gene from a specific locus within the genome of the transgenic avian subject, involves in some embodiments, the provision of the CRISPR/Cas system that includes specific gRNA.

In some embodiments, the at least one reporter gene in the third nucleic acid sequence, the fourth nucleic acid, or both, used by the method of the invention, is flanked with two arms that are homologous or show homology or identity of at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% to the whole, entire, or complete at least one nucleic acid sequence comprised within the target loci within the gender chromosome Z or W, that serves as the integration site to facilitate specific integration via HDR, or any value and range therebetween. Each possibility represents a separate embodiment of the invention. In some embodiments, the at least one reporter gene in the third nucleic acid sequence used by the method of the invention, is flanked with two arms that are homologous or show 100% homology or identity to the whole, entire, or complete at least one nucleic acid sequence comprised within the target loci within the gender chromosome Z, that serves as the integration site to facilitate specific integration via HDR.

In some embodiments, a target sequence is also referred to herein as at least one “proto-spacer” that is recognized by the “spacer” sequence that is part of the gRNA used by the invention and provided by the first nucleic acid sequence or the second nucleic acid.

The term “Homologous arms”, as used herein refers to HDR templates introduced into specific vectors or viruses, used to create specific mutations or insertion of new elements into a gene, that possess a certain amount of homology surrounding the target sequence to be modified (depending on which PEN is used). In some embodiments, where CRISPR is used as a PEN, the arms sequences (left, upstream and right, downstream) comprise between 10 to 5,000 bp, between 50 to 1,000 bp, or between 100 to 500 bp. Each possibility represents a separate embodiment of the invention. in some embodiments, where CRISPR is used as a PEN, the arms sequences (left, upstream and right, downstream) comprise at least: 50 bp, 100 bp, 200 bp, 300 bp, 400 bp, 500 bp, 600 bp, 700 bp, 800 bp, 900 bp, 1000 bp, 2,000 bp, 3,500 bp or 5,000 bp, or any value and range therebetween. Each possibility represents a separate embodiment of the invention.

In some embodiments, the targeting sequence within the gRNA encoded by the first nucleic acid sequence or the second nucleic acid sequence provided by the methods and kits of the invention, also referred to herein as the “spacer” sequence, exhibits homology or identity of at least: 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% to the entire, complete or whole at least one nucleic acid sequence comprised within the target loci within the gender chromosome Z, or W, referred to herein as the “proto-spacer”, or any value and range therebetween. Each possibility represents a separate embodiment of the invention. In some embodiments, the targeting sequence within the gRNA encoded by the first nucleic acid sequence or the second nucleic acid sequence provided by the methods and kits of the invention, also referred to herein as the “spacer” sequence, exhibits 100% homology or identity to the entire, complete or whole at least one nucleic acid sequence comprised within the target loci within the gender chromosome Z or W, referred to herein as the “proto-spacer”. Each possibility represents a separate embodiment of the invention.

In some embodiments, the at least one reporter gene may be inserted and thereby integrated into at least one non-coding region of the target gender chromosome. Such approach avoids the disruption of genes that may be required for development and maturation of the unhatched embryo.

In some embodiments, any one of the first nucleic acid sequence, the second nucleic acid sequence, third nucleic acid, and the fourth nucleic acid sequence is inserted and/or incorporated into at least one non-coding region of the target region of a target gender chromosome.

“Non-coding region” as used herein, refers to components of an organism's DNA that do not encode protein sequences. Some noncoding DNA region is transcribed into functional non-coding RNA molecules, other functions of noncoding DNA regions include the transcriptional and translational regulation of protein-coding sequences, scaffold attachment regions, origins of DNA replication, centromeres and telomeres. The hypothesized non-functional portion (or DNA of unknown function) has often been referred to as “junk DNA”.

In some embodiments, when genetic loci of an avian cell., e.g., a zygote, has been targeted and/or transfected with exogenous sequences, specifically, as described, it is desirable to use such a cell to generate a transgenic subject therefrom. For such a procedure, following the introduction of the targeting construct into the embryonic stem (ES) cells, the cells may be plated onto a feeder layer in an appropriate medium, for example, DMEM supplemented with growth factors and cytokines, fetal bovine serum and antibiotics. The embryonic stem cells may have a single targeted locus (heterozygotic) or both loci targeted (homozygotic). Cells containing the construct may be detected by employing a selective medium and after sufficient time for colonies to grow, colonies may be picked and analyzed for the occurrence of gene targeting. In some embodiments, PCR is applied to verify the integration of the desired exogenous sequences into the target loci, using primers within and outside the construct sequence. Colonies which show gene targeting may then be used for injection into avian embryos. The ES cells can then be trypsinized and the modified cells can be injected through an opening made in the side of the egg. After sealing the eggs, the eggs can be incubated under appropriate conditions until hatching. Newly hatched avian can be tested for the presence of the target construct sequences, for example by examining a biological sample thereof, e.g., a blood sample. After the avian have reached maturity, they are bred, and their progeny may be examined to determine whether the exogenous integrated sequences are transmitted through the germ line.

Chimeric avian subjects are generated which are derived in part from the modified embryonic stem cells or zygote cells, capable of transmitting the genetic modifications through the germ line. Mating avian strains containing exogenous sequences, specifically, as disclosed herein, with strains in which the avian wild type loci, or portions thereof, is restored, should result in progenies displaying an in-ovo detectable gender.

Still further, transgenic avian subjects can also be produced by other methods, some of which are discussed below. Among the avian cells suitable for transformation for generating transgenic animals are primordial germ cells (PGC), sperm cells and zygote cells (including embryonic stem cells). Sperm cells can be transformed with DNA constructs by any suitable method, including electroporation, microparticle bombardment, lipofection and the like. The sperm can be used for artificial insemination of avian. Progeny of the inseminated avian can be examined for the exogenous sequence as described above.

Alternatively, primordial germ cells may be isolated from avian eggs, transfected with the exogenous gene of the invention, e.g., comprises at least one reporter gene, by any appropriate method, and transferred or inserted into new embryos, where they can become incorporated into the developing gonads. Hatched avian and their progeny can be examined for the presence of the exogenous gene sequence, e.g., detectable signal derived from the at least one reporter gene of the exogenous gene, as described by the invention.

In some embodiments, dispersed blastodermal cells isolated from eggs can be transfected by any appropriate means with the exogenous gene sequence, or portions thereof, integrated to the gender specific chromosome Z, W, or both, followed by injection into the subgerminal cavity of intact eggs. Hatched avian subjects and their progeny may be examined for the presence of the exogenous gene sequence, e.g., detectable signal derived from the at least one reporter gene of the exogenous gene, as described by the invention.

Chicken primordial germ cells (PGCs) are the precursors for ova and spermatozoa. Thus, in some embodiments, the invention provides the production of at least one transgenic chicken via a germline transmission system using PGCs co-transfected with an exogenous gene with the CRISPR/Cas gRNA construct that directs the integration of the exogenous gene into the gender chromosome Z, W, or both. PGCs are sorted and transferred into the bloodstream of 2.5-day recipient embryos for germline transmission.

PGCs are the precursors of gametes and show unique migration activity during early embryogenesis in birds, when compared to mammals. In avian embryos, PGCs first arise from the epiblast and migrate to the hypoblast of the germinal crescent at stage 4 (HH), approximately 18-19 h after incubation. Between stages 10 and 12 (HH), PGCs move from the germinal crescent into the bloodstream and migrate through the circulatory system until they reach the genital ridges and colonize the developing gonads. This contrasts with mammals in which PGCs migrate through the embryonic tissues to reach the developing gonads. It is this unique feature of avian PGC migration through the blood stream that has facilitated the major advance in the genetic modification of chickens.

In some embodiments, the “preparation of transgenic avian animal” refers to a multi-step method involving genetic engineering techniques for production of chicken with genomic modifications wherein a) PGCs are isolated from the blood of two days-old chick embryos; b) a transgene construct is incorporated into cultured PGCs, for example, by using lentiviral system, Piggybac transposon vectors, TALENS or CRISPR/Cas techniques; (c) transgenic PGCs are identified and injected into the circulatory system of embryos and migrate to the developing gonads; d) recipient embryos are incubated at 37° C. until hatching (d) hatched males are reared to sexual maturity and crossed with wild-type hens (e) offspring are screened to identify those derived from the transgenic PGCs.

In another embodiments, the invention relates to an avian transgenic animal or subject comprising, in at least one cell thereof, at least one exogenous reporter gene integrated into at least one position or location (also referred to herein as locus) in gender chromosome Z.

In another embodiments, the invention relates to an avian transgenic animal or subject comprising, in at least one cell thereof, at least one exogenous reporter gene integrated into at least one position or location (also referred to herein as locus) in gender chromosome W.

In another embodiments, the invention relates to an avian transgenic animal or subject comprising, in at least one cell thereof, at least one exogenous reporter gene integrated into at least one position or location (also referred to herein as locus) in both gender chromosomes Z.

As used herein, the term “avian” refers to any species derived from birds characterized by feathers, toothless beaked jaws, the laying of hard-shelled eggs, a high metabolic rate, a four-chambered heart, and a lightweight but strong skeleton. Avian species includes, without limitation, chicken, quail, turkey, duck, Gallinacea sp., goose, pheasant and other fowl. The term “hen” includes all females of the avian species. A “transgenic avian” generally refers to an avian that has had a heterologous DNA sequence, or one or more additional DNA sequences normally endogenous to the avian (collectively referred to herein as “transgenes”) chromosomally integrated into the germ cells of the avian. As a result of such transfer and integration, the transferred sequence may be transmitted through germ cells to the offspring of a transgenic avian. The transgenic avian (including its progeny) also have the transgene integrated into the gender chromosomes of somatic cells.

In some embodiments, the gRNA used by the method, as well by kit of the invention (described herein after) may comprise at least one CRISPR RNA (crRNA) and at least one trans-activating crRNA (tracrRNA).

In some embodiments the kit of the invention may comprise nucleic acid sequence encoding the at least one gRNA. Such nucleic acid sequence may comprise a CRISPR array comprising at least one spacer sequence that targets and is therefore homolog or identical to at least one protospacer in a target genomic DNA sequence. It should be noted that the nucleic acid sequence may further comprise a sequence encoding at least one tracrRNA.

In some embodiments the CRISPR array according to the present disclosure comprises at least one spacer and at least one repeat. In yet another embodiment, the invention further encompasses the option of providing a pre-crRNA that can be processed to several final gRNA products that may target identical or different targets.

In some embodiments, the crRNA comprised within the gRNA of the invention may be a single-stranded ribonucleic acid (ssRNA) sequence complementary to a target genomic DNA sequence. In some specific embodiments, the target genomic DNA sequence may be located immediately upstream of a protospacer adjacent motif (PAM) sequence and further.

As indicated above, the genomic DNA sequence targeted by the gRNA of the kit of the invention is located immediately upstream to a PAM sequence. In some embodiments, such PAM sequence may be of the nucleic acid sequence NGG.

In some embodiments, the PAM sequence referred to by the invention may comprise N, that is any nucleotide, specifically, any one of Adenine (A), Guanine (G), Cytosine (C) or Thymine (T). In yet some further embodiments the PAM sequence according to the invention is composed of A, G, C, or T and two Guanines.

According to some embodiments, the polynucleotide encoding the gRNA of the invention may comprise at least one spacer and optionally, at least one repeat. In some embodiments, the DNA encoding the gRNA of the invention may comprise at least: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200 or more spacers, or any value and range therebetween. Each possibility represents a separate embodiment of the invention. In some embodiments, each spacer is located between two repeats. It should be further understood that the spacers of the nucleic acid sequence encoding the gRNA of the invention may be either identical or different spacers. In some embodiments, these spacers may target either an identical or different target genomic DNA. In some embodiments, such spacer may target at least: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more target genomic DNA sequence, or any value and range therebetween. Each possibility represents a separate embodiment of the invention These target sequences may be derived from a single locus or alternatively, from several target loci.

In some embodiments, a crRNA comprises or consists of 20, 21, 22, 23, 24, 25, 26, 27, 28, 29, 30, 31, 32, 33, 34, 35, 36, 37, 38, 39 or 40 nt of the spacer (targeting) sequence followed by 19-36 nt of repeat sequence. In specific and non-limiting embodiments, the targeting spacer may comprise or consist of a segment that targets any one of the genomic DNA sequence for which representative spacer sequences are indicated herein.

By “fragments or peptides” it is meant a fraction of a protein, in some embodiments, the Cas or RFP (described herein after) molecules. A “fragment” of a molecule, such as any of the amino acid sequences of the present invention, is meant to refer to any amino acid subset of the Cas or RFP molecules. This may also include “variants” or “derivatives” thereof. A “peptide” is meant to refer to a particular amino acid subset having functional activity. By “functional” is meant having the same biological function, for example, having the ability to perform gene editing as the Cas or as producing a detectable signal as the RFP.

It should be appreciated that the invention encompasses any variant or derivative of the RFP or Cas molecules of the invention and any polypeptides that are substantially identical or homologue. The term “derivative” is used to define amino acid sequences (polypeptide), with any insertions, deletions, substitutions and modifications to the amino acid sequences (polypeptide) that do not alter the activity of the original polypeptides. In this connection, a derivative or fragment of the Cas or RFP molecules of the invention may be any derivative or fragment of the Cas or RFP molecules that do not reduce or alter the activity of the Cas9 or RFP molecules. By the term “derivative” it is also referred to homologues, variants and analogues thereof. Proteins orthologs or homologues having a sequence homology or identity to the proteins of interest in accordance with the invention, specifically that may share at least: 50%, 60%, 65%, 70%, 75%, 80%, 85%, 90%, 91%, 92%, 93%, 94%, 95%, 96%, 97%, 98%, 99%, or 100%, or ant value and range therebetween. Each possibility represents a separate embodiment of the invention. Specifically, homologs that comprise or consists of an amino acid sequence that is identical in at least 50%, 60%, 65%, 70%, 75%, 80%, 85%, 90%, 91%, 92%, 93%, 94%, 95%, 96%, 97%, 98%, 99%, or higher to a sequence, or any value and range therebetween. Each possibility represents a separate embodiment of the invention.

In some embodiments, derivatives refer to polypeptides, which differ from the polypeptides specifically defined in the present invention by insertions, deletions or substitutions of amino acid residues. It should be appreciated that by the terms “insertion/s”, “deletion/s” or “substitution/s”, as used herein it is meant any addition, deletion or replacement, respectively, of amino acid residues to the polypeptides disclosed by the invention, of between 1 to 50 amino acid residues, between 20 to 1 amino acid residues, and specifically, between 1 to 10 amino acid residues. More particularly, insertion/s, deletion/s or substitution/s may be of any one of 1, 2, 3, 4, 5, 6, 7, 8, 9, or 10 amino acids. It should be noted that the insertion/s, deletion/s or substitution/s encompassed by the invention may occur in any position of the modified peptide, as well as in any of the N′ or C′ termini thereof.

With respect to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologues, and alleles of the invention.

For example, substitutions may be made wherein an aliphatic amino acid (G, A, I, L, or V) is substituted with another member of the group, or substitution such as the substitution of one polar residue for another, such as arginine for lysine, glutamic for aspartic acid, or glutamine for asparagine. Each of the following eight groups contains other exemplary amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M).

Amino acid “substitutions” are the result of replacing one amino acid with another amino acid having similar structural and/or chemical properties, i.e., conservative amino acid replacements. Amino acid substitutions may be made on the basis of similarity in polarity, charge, solubility, hydrophobicity, hydrophilicity, and/or the amphipathic nature of the residues involved. For example, nonpolar “hydrophobic” amino acids are selected from the group consisting of Valine (V), Isoleucine (I), Leucine (L), Methionine (M), Phenylalanine (F), Tryptophan (W), Cysteine (C), Alanine (A), Tyrosine (Y), Histidine (H), Threonine (T), Serine (S), Proline (P), Glycine (G), Arginine (R) and Lysine (K); “polar” amino acids are selected from the group consisting of Arginine (R), Lysine (K), Aspartic acid (D), Glutamic acid (E), Asparagine (N), Glutamine (Q); “positively charged” amino acids are selected form the group consisting of Arginine (R), Lysine (K) and Histidine (H) and wherein “acidic” amino acids are selected from the group consisting of Aspartic acid (D), Asparagine (N), Glutamic acid (E) and Glutamine (Q).

As used herein, a variant or a derivative has at least: 80%, 85%, 90%, 95%, 96%, 97%, 98%, or 99% sequence similarity or identity at the amino acid level, with the protein of interest, such as the protein product of the at least one reporter gene of the invention, or any value and range therebetween. Each possibility represents a separate embodiment of the invention.

It should be appreciated that in certain embodiments, the oligonucleotide/s or polynucleotide/s used by the kit/s and method/s of the invention are isolated and/or purified molecules. As used herein, “isolated” or “purified” when used in reference to a nucleic acid means that a naturally occurring sequence has been removed from its normal cellular (e.g., chromosomal) environment or is synthesized in a non-natural environment (e.g., artificially synthesized). Thus, an “isolated” or “purified” sequence may be in a cell-free solution or placed in a different cellular environment. The term “purified” does not imply that the sequence is the only nucleotide present, but that it is essentially free (about 90-95% pure) of non-nucleotide material naturally associated with it, and thus is distinguished from isolated chromosomes.

All scientific and technical terms used herein have meanings commonly used in the art unless otherwise specified. The definitions provided herein are to facilitate understanding of certain terms used frequently herein and are not meant to limit the scope of the present disclosure.

Before specific aspects and embodiments of the invention are described in detail, it is to be understood that this invention is not limited to particular methods, and experimental conditions described, as such methods and conditions may vary. It is also to be understood that the terminology used herein is for the purpose of describing particular embodiments only, and is not intended to be limiting, since the scope of the present invention will be limited only by the appended claims.

As used in this specification and the appended claims, the singular forms “a”, “an”, and “the” include plural references unless the context clearly dictates otherwise. Thus, for example, references to “a method” includes one or more methods, and/or steps of the type described herein and/or which will become apparent to those persons skilled in the art upon reading this disclosure and so forth.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps. More specifically, the terms “comprises”, “comprising”, “includes”, “including”, “having” and their conjugates mean “including but not limited to”. This term encompasses the terms “consisting of” and “consisting essentially of”. The phrase “consisting essentially of” means that the composition or method may include additional ingredients and/or steps, but only if the additional ingredients and/or steps do not materially alter the basic and novel characteristics of the claimed composition or method.

The term “about” as used herein indicates values that may deviate up to 1%, more specifically 5%, more specifically 10%, more specifically 15%, and in some cases up to 20% higher or lower than the value referred to, the deviation range including integer values, and, if applicable, non-integer values as well, constituting a continuous range. As used herein the term “about” refers to ±10%.

It should be noted that various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible sub ranges as well as individual numerical values within that range. For example, description of a range such as from 1 to 6 should be considered to have specifically disclosed sub ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range. Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals there between.

The following examples are put forth so as to provide those of ordinary skill in the art with a complete disclosure and description of how to make and use the methods and compositions of the invention and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

The examples are representative of techniques employed by the inventors in carrying out aspects of the present invention. It should be appreciated that while these techniques are exemplary of preferred embodiments for the practice of the invention, those of skill in the art, in light of the present disclosure, will recognize that numerous modifications can be made without departing from the spirit and intended scope of the invention.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub combination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

Disclosed and described, it is to be understood that this invention is not limited to the particular examples, methods steps, and compositions disclosed herein as such methods steps and compositions may vary somewhat. It is also to be understood that the terminology used herein is used for the purpose of describing particular embodiments only and not intended to be limiting since the scope of the present invention will be limited only by the appended claims and equivalents thereof.

It must be noted that, as used in this specification and the appended claims, the singular forms “a”, “an” and “the” include plural referents unless the content clearly dictates otherwise.

### EXAMPLES

Generally, the nomenclature used herein, and the laboratory procedures utilized in the present invention include chemical, molecular, biochemical, and cell biology techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); “Cell Biology: A Laboratory Handbook”, Volumes I-III Cellis, J. E., ed. (1994); The Organic Chemistry of Biological Pathways by John McMurry and Tadhg Begley (Roberts and Company, 2005); Organic Chemistry of Enzyme-Catalyzed Reactions by Richard Silverman (Academic Press, 2002); Organic Chemistry (6th Edition) by Leroy “Skip” G Wade; Organic Chemistry by T. W. Graham Solomons and, Craig Fryhle; and “Functional genomics using CRISPR-Cas systems, compositions, methods, screens and applications thereof”, U.S. patent application Ser. No. 15/141,348 to the Whitehead Institute for Biomedical Research Massachusetts Institute of Technology Broad Institute Inc.

### Example 1

### Obtaining an Avian Transgenic Cell

The process includes knocking-in a reporter gene (CMV-RFP) to the designated genome loci, which is described hereinbelow.

Preparation of gRNA Duplex:

Components: Z-specific gRNA oligo (ChrZgRNA sequence: 5′-GUAAUACAGAGCUAAACCAG-3′; SEQ ID NO: 30); tracrRNA (IDT cat #1072533); and Nuclease-Free Duplex Buffer (IDT cat #11-01-03-01).

RNA oligos are resuspend in Nuclease-Free Duplex Buffer to a final concentration of 100 μM (Micromolar) (for 10 nM (nanomolar) gRNA use 100 μl of Nuclease free buffer). Z-specific gRNA and tracrRNA are mixed in equimolar concentrations to create a final duplex concentration of 1 μM from each oligo (the duplex can be stored in −20° C. for multiple uses). Duplexes are heated at 95° C. for 5 min, and thereafter are cooled to room temperature (20-25 ° C.).

### Preparation of RNP Complex and Transfection:

Components: HiFi Cas9 Nuclease V3 (IDT cat #1081061); Opti-MEM reduced serum media (Thermo cat #11058021); LIPOFECTAMINE™ 3000™ Transfection Reagent (Thermo cat #L3000008); and Z-chromosome arms: RFP Donor plasmid/DNA block.

Cas9 enzyme is diluted to a working concentration of 1 μM in OptiMEM. Six (6) μl of gRNA:tracrRNA duplex, 2 μl of diluted Hifi Cas9 nuclease, 1.2 μl P3000™ reagent (from LIPOFECTAMINE™ 3000™ kit), are mixed. The mixture is then added to 40.8 μl of Opti-MEM to obtain a final volume of 50 μl. The mixture is incubated at room temperature for 5 min to assemble the RNP complexes. Five hundred (500) ng of donor plasmid/DNA block are added and subsequently incubated for an additional 5 min. Fifty (50) μl of RNP complex are mixed with 2 μl of LIPOFECTAMINE™ 3000 reagent and added to 48 μl of Opti-MEM to a final volume of 100 μl. The mixture is incubated at room temperature (20-25° C.) for 20 min to form transfection complexes. Thereafter, the transfection mix is added to cells. The cells are incubated for 48 hrs in a tissue culture incubator (39° C., 5% CO₂).

Transfected cells are visualized for RFP via fluorescence microscopy within 24-48 hrs. Stable transformation can be detected following 7-14 days. Validation of genome insertion via PCR/sequencing is described hereinbelow.

### Example 2

### Obtaining a Transgenic Embryo/Organism

In order to generate transgenic chick, primordial germ cells (PGCs) are extracted, manipulated and retrieved to another embryo for the purpose of germ line chimera generation.

### Collection of 18 Hamburger Hamilton (HH) Stage Embryonic Blood for the Purpose of PGC Isolation

Fertilized eggs are incubated in 39° C. incubator for 72 hrs. Following the 72 hrs incubation, eggs are removed from the incubator, and each egg is punctured using a 19 G needle. Three (3) to 5 ml of albumin is drawn from each egg. A “window” is opened in each egg by carefully removing a part of shell. A heated 5 μl pointed capillary is used to cause controlled bleeding from the embryo's vascular system. Up to 5 μl of embryonic blood is collected and deposited into 48-wells plate (48 W) filled with Avian DMEM media. After 1-2 weeks, red blood cells die and PGCs are visible. Fresh medium is replenished every 3 or 4 day. When PGCs reach confluency in the 48 W, they are transferred to a 24-well plate. Medium is replaced every 3 or 4 day. When cell number exceeds 2×10⁵ cells/ml, cells are subsequently passaged to reach a density of 1-1.5×10⁵ cells/ml.

Transfection of Isolated PGCs with CMV-RFP

Cas9 enzyme is diluted to a working concentration (1 μM) in OptiMEM. Six (6) μl of gRNA:tracrRNA duplex, 2 μ of diluted Hifi Cas9 nuclease, and 1.2 μl P3000™ reagent (from LIPOFECTAMINE™ 3000™ kit) are mixed. The mixture is then added to 40.8 μl of Opti-MEM to obtain a final volume of 50 μl. The mixture is incubated at room temperature for 5 min to assemble the RNP complexes. Five hundred (500) ng of donor plasmid/DNA block are added and subsequently incubated for an additional 5 min. Fifty (50) μl of RNP complex are mixed with 2 μl of LIPOFECTAMINE™ 3000 reagent and added to 48 μl of Opti-MEM to a final volume of 100 μl. The mixture is incubated at room temperature (20-25° C.) for 20 min to form transfection complexes. Thereafter, the transfection mix is added to 5×10⁴−1×10⁵ cells/well. The cells are incubated for 4-6 hrs in a tissue culture incubator (39° C., 5% CO₂). Then, transfection mix is removed from the cells and avian-DMEM medium is added to each well comprising the transfected PGCs.

Transfected PGCs are visualized for RFP via fluorescent microscopy within 24-48 hrs. PGCs are subcultured, and further isolated from red blood cells using cell sorting. Stable transformation can be detected following 7-14 days. Validation of genome insertion via PCR/sequencing is described hereinbelow.

Microinjection of RFP-PGCs into Recipient Embryo

Fertilized eggs are incubated in an incubator set at 39° C. for 72 hrs. Following the 72 hrs, eggs are removed from the incubator, and each egg is punctured using a 19 G needle. Three (3) to 5 ml of albumin is drawn from each egg. A “window” is opened in each egg by carefully removing a part of shell. Up to 2 μl of RFP-PGCs are injected directly into the dorsal aorta of ˜18 HH chick embryo (>500 cells/μl). The shell “window” is sealed using parafilm. The eggs are subsequently incubated until hatching, e.g., for additional 18 days, so as to receive germline chimeras. The chicks comprising the germline chimeras are further reared and are cross breed to produce stably transformed RFP chicks.

### Example 3

### Validation of Genome Editing (GE) Event

### Validation of Genome Cleavage Using T7EI Enzyme Assay

Components: GENEART™ Genomic Cleavage Detection Kit (Thermo cat#A24372); gRNA: Cas9 transfected DF1 cells (an ATCC cell line #CRL12203); and detection primers for cleavage with ChrZgRNA: Forward: 5′-GCGGAGGGAGATGAAACA-3′ (SEQ ID NO: 31), and Reverse: 5′-AAGGGATCACACAGCTCAAG-3′ (SEQ ID NO: 32).

Hundred (100) μl of cell lysis buffer (GeneArt Kit) are mixed with 4 μl of protein degrader (GeneArt Kit), in a microcentrifuge tube. Culture media is removed from transfected cells, which are then added with 100 μl of the cell lysis mix. The cells are then incubated at room temperature for 5 min. Lysed cells are then collected and transfer into a PCR (0.2 μl) tube. The lysed cells are placed in a thermo-cycler and processed according to the following program: 68° C. for 15 min, 95° C. for 10 min, and hold on 4° C. Thereafter, an amplification reaction is set according to the following table:

| Ingredient | Volume | |
|---|---|---|
| Cell lysate | 2 | μl |
| F primer (SEQ ID NO: 31) | 1 | μl |
| R primer (SEQ ID NO: 32) | 1 | μl |
| AMPLITAQ GOLD ® 360 Master | 25 | μl |
| H₂O | 21 | μl |
| TOTAL | 50 | μl |

The amplification reaction(s) are placed in a thermo-cycler set for the following program:

| | | |
|---|---|---|
| Temperature | Time | No. of cycles |

| | | | | |
|---|---|---|---|---|
| 95° | C. | 10 | min | ×1 |
| 95° | C. | 30 | sec | ×40 |
| 54° | C. | 30 | sec | |
| 72° | C. | 30 | sec | |
| 72° | C. | 7 | min | ×1 |

| | | |
|---|---|---|
| 4° | C. | Hold |

Upon amplification, 3 μl of each PCR product are mixed with 10 μl water, and are then loaded on a 2% (w/v) agarose gel. Electrophoresis is initiated in 100 V for 30 min.

A single band of 251 bp, is indicative of the cells comprising the desired sequence. If so, a denaturation and re-annealing step is performed. Samples can also be stored in −20° C. for later use.

Three (3) μl of the PCR product are mixed with 1 μl 10× detection reaction buffer in a PCR tube. The volume is adjusted to 9 μl by adding 5 μl of water, and the resulting reaction mix is placed in a thermal cycler set for the following program:

| Temperature | Time |
|---|---|
| 95° C. | 5 min |
| Cooldown from | If possible, cooldown |
| 95° C. to 25° C. | at the rate of −0.1° C./sec |
| 4° C. | Hold |

Each sample is added with 1 μl of a detection enzyme (including tests and controls), and is incubate at 37° C. for 1 hr. Post incubation, load the entire sample volume (e.g., 10 μl) on a 2% (w/v) agarose gel and initiate electrophoresis as mentioned above. The gel is then viewed using a UV transilluminator. A digested band in addition to the estimated size band as an evidence to the genome cleavage caused by the gRNA/Cas9, is expected.

Validation of Transgene Knock-In

In general, validation of successful knock-in is performed by amplifying (using PCR) the genome loci harboring the site manipulation. The PCR primers include one oligo complementary/with homology to the transgene sequence, and a second oligo complementary/with homology to the adjacent genomic sequence.

### Validation of Knock-In Success by Amplifying the “Left” Region of Insertion

Medium is aspirated from the transfected cells' well. Cells are washed briefly with PBS×1. Two hundred (200) μl (for a 48 W) of universal digestion buffer (BIO-BASIC cat#AT4781) are applied to the cells, which in turn are and incubated at room temp for 5 min. The lysed cells and transferred to an Eppendorf tube, and are incubated in 68° C. for 15 min followed by another incubation in 95° C. for 10 min and hold on 4° C. The resulting cell lysis mixture is diluted 1:10 (v/v). A PCR reaction mix is prepared as follows (for each cell mixture): 1 μl of Forward primer (5′-TCCCATGGCATTGATCTGT-3′; SEQ ID NO: 33), 1 μl Reverse primer (5′-CTATCCACGCCCATTGATGTA-3′; SEQ ID NO: 34), 1.5 μl of the cell lysis mixture, 9 μl of H₂O, and 12.5 μl 2× hy-Tiger PCR mix (Hy-Labs #EZ-2031). The amplification reaction(s) are placed in a thermo-cycler set for the following program:

| | | |
|---|---|---|
| Temperature | Time | No. of cycles |

| | | | | |
|---|---|---|---|---|
| 95° | C. | 10 | min | ×1 |
| 95° | C. | 30 | sec | ×40 |
| 55° | C. | 30 | sec | |
| 72° | C. | 60 | sec | |
| 72° | C. | 7 | min | ×1 |

| | | |
|---|---|---|
| 4° | C. | Hold |

Ten (10) μl of each PCR product is loaded on a 1.2% (w/v) agarose gel, wherein the expected product size is a band of about 1.5 Kb.

The PCR product is then excised from gel, and is subsequently extracted and purified (Geneaid Gel/PCR DNA fragments extraction kit cat#DF100, according to Kit's protocol). The purified PCR product is sequenced so as to ensure sequence identity (e.g., by sanger sequencing).

### Example 4

### Crossing a Transgenic Male and Female, Obtaining the Progeny, and Subsequent Rearing

The use of founder lines is a common practice in chick breeding. The lines A & B are used to generate the male for the desired cross. Lines C & D are used to obtain the female hen for the desired cross.

In order to obtain ABCD chick with RFP detectable egg, the generation of Z^{RFP}/Z^{RFP} C males is needed for the subsequent breeding.

Generation of Male Z^{RFP}/Z^{RFP} C×D Female

Microinject Z^{RFP} PGCs are prepared from C line to the corresponded recipient embryos. The microinjected embryos are incubated for another 18 days until hatching. Germ line chimeras from each line are crossed to obtain Z^{RFP} male chicks. Hatched chicks are screened for the presence of RFP using a UV lamp. RFP-harboring male chicks are grown to sexual maturity. Male C (Z^{RFP}/Z^{RFP}) X female D (W/Z) are crossed. Thereafter, a female CD (W/Z^{RFP}) is sought for subsequent breeding with AB males.

For sex selection purposes, the PGCs of line A and B are edited for knock-in of Cas9 sequence (regulated by the appropriate promoter), to ^{their} Z chromosome. The Z^{cas9} PGCs are transferred to the appropriate recipient host (each PGC line to the corresponded recipient embryo) in order to obtain germ line chimera. Germline chimeras are crossbred to provide z^{Cas9}/Z^{Cas9} male chicks. PGCs of line C (males) are edited to accommodate the desired gRNA sequence on their chromosome Z. The Z^{gRNA} PGCs are injected to C line embryos in order to provide Z^{gRNA} chimera. Chimeric chicks of line C are further crossbred to provide Z^{gRNA}/Z^{gRNA} males. The edited C line chicks are then ready for crossbreeding with line D females to provide W/Zg^{RNA} CD females. The cross between A×B Z^{Cas9}/Z^{Cas9} males with C×D W/Z^{gRNA} females allows in-ovo selection against males due to the presence of both Cas9 and lethal gRNA in AB×CD males.

### Example 5

### Identification of Z^{RPF} Positive Chick Embryos

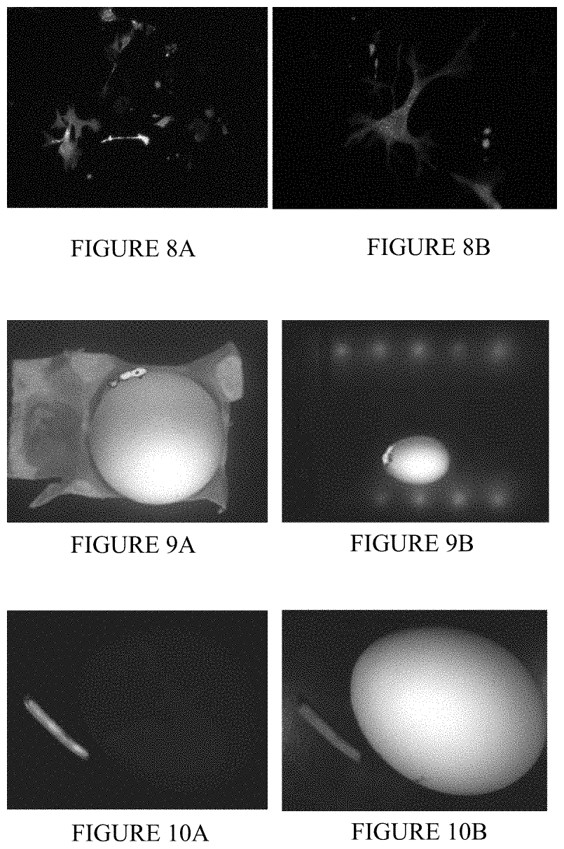

The identification of RFP expression in Z^{RFP} primary chick embryo fibroblast was performed. Primary fibroblasts were obtained by preparation of primary culture from embryonic tail tissue. The primary cells were cultured and plated in 48 W at the confluency of 50-70% after which, the cells were treated as described above. Cells were cultured for additional 14 days following genome editing manipulation, after which they were examined using a fluorescent microscope with RFP filter (wavelength 550-650 nm). Populations of red-fluorescently labeled cells were identified (FIG. 8). Therefore, this example shows that chicken embryonic cells can be gene edited so as to include a reporter gene, e.g., RFP encoding sequence, integrated into a gender Z chromosome, specifically the gender chromosome Z.

### Example 6

Selection of reporter gene for visual gender identification in poultry

For observation of luciferase activity, luciferin was injected subcutaneously to luciferase-expressing transgenic mice, tails and ears were then excised and introduced through a 5 mm hole in the eggshell of an unfertilized egg. As shown (FIG. 9), the luciferase detectable signal is clearly observed in tail and ear samples (FIGS. 9A-10B) through the eggshell. The inventors therefore next examined the feasibility of inducing luciferase reaction in-ovo. Therefore, ears and tails of luciferase-expressing transgenic mice were excised, introduced through a hole into a fertilized egg that carry a 10-days old chicken embryo and luciferin was subsequently injected. As was clearly shown, an in-ovo luciferase reaction successfully resulted in a detectable signal that was able to penetrate the eggshell.

On the other hand, similar experiments performed using GFP as the reporter gene, clearly indicated that GFP signal is not detectable following incorporation of tails and ears of GFP-expressing transgenic mice into chicken embryo (FIGS. 10A-10B).

### Example 7

### Optical Fluorescent System

In order to demonstrate the feasibility of visually identify gender of in-ovo poultry, the use of green fluorescence as compared to red fluorescence reporter genes was evaluated.

The autofluorescence of complete eggs or of eggs separated into egg white, egg yolk and shell was determined using a green laser or a red laser. Comparison of an empty egg (shell) to a complete egg indicates no significant difference in autofluorescence level (data not shown).

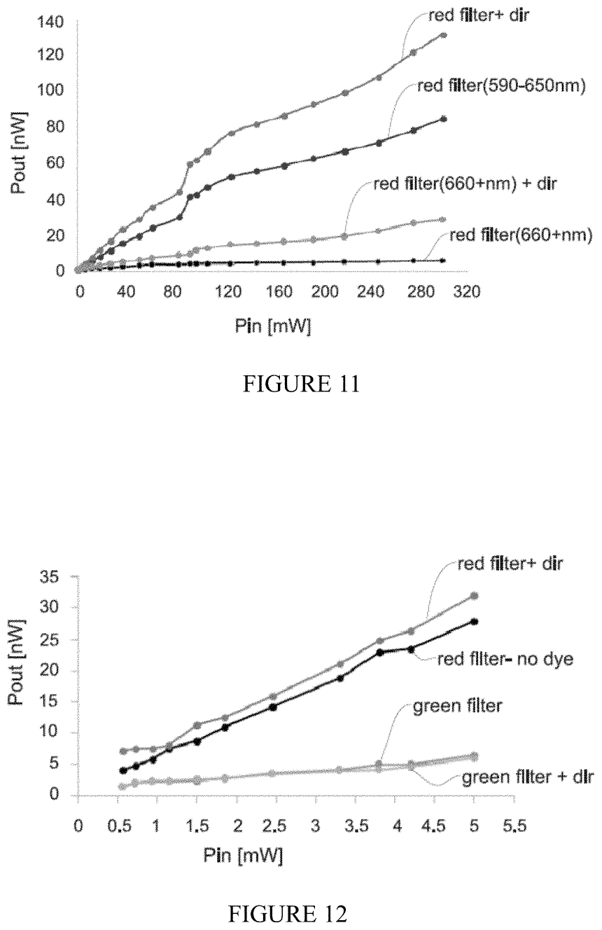

The use of a green laser (532 nm) together with a red filter A (590-650 nm) or a red filter B (+660 nm) with or without addition of dir (10 μM) into a complete egg was assayed and is summarized in FIG. 11. It appears that above 80 mW, the fluorescent emission of the dye is detectable using red filter A while by using the red filter B (+660 nm), only a modest difference was observed.

The use of a red laser (632.8 nm) together with a red filter (+660 nm) or a green filter (540-580 nm) with or without addition of dir (10 μM) into a complete egg was assayed and is summarized in FIG. 12. Almost no intensity difference was shown with or without fluorescent dye. All of the experiments were repeated using only the eggshell. Similar results were observed (data not shown). These observations demonstrate the feasibility of detecting RFP through the eggshell.

### Example 8

Fluorescence of RFP or GFP Transfected Cells into Eggs

To illustrate the feasibility of in-ovo detection of RFP expressing cells in an embryo, the inventors next examined if cells transfected with a fluorescent protein, specifically, RFP or GFP, can be detected upon injection thereof into a complete egg.

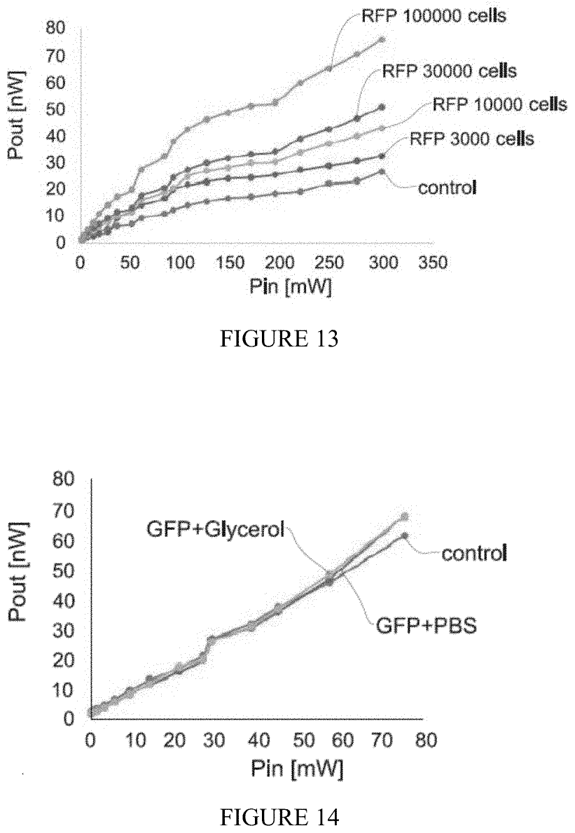

More specifically, HEK cells were transfected RFP vector. The fluorescence of RFP transfected cells was examined following excitation. FIG. 13 shows the measured fluorescence intensity using different concentrations of RFP-expressing cells when the egg was excited at the center. The correlation between the RFP concentration and the intensity on the detector was clearly observed.

The filter of +500 ran was examined with or without the presence of GFP-expressing cells. It appears that GFP-expressing cells could not be detected in any given concentration (1000, 3000, 10,000, and 30,000 cells), as illustrated by FIG. 14 for example with 30,000 GFP expressing cells. The same results were obtained using three different filters: +500 nm, 530-550 nm and 540-580 nm.

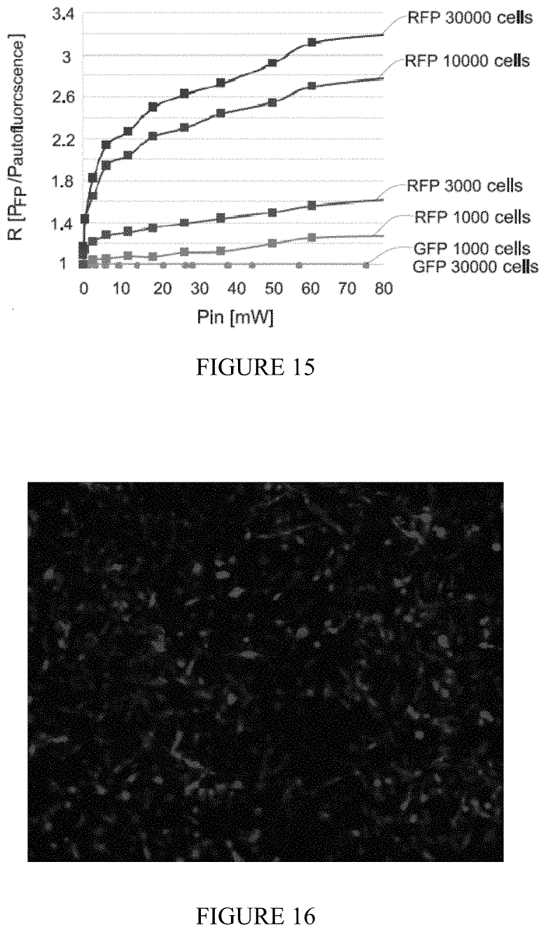

To further evaluate the surprising advantage of using RFP as a reporter gene, the fluorescence of GFP and RFP-expressing cells were finally compared (FIG. 15).

### Example 9

Integration of the RFP reporter gene into the Z chromosome of female chicken cell line

Female chicken cell line was co-transfected with two plasmids e.g., pDsRed containing the chicken chromosome Z Left and Right arms and the plasmid containing the CMV- spCas9-H1-gRNA. Transfected cells were then exited with red fluorescent. As can be seen in FIG. 16 the transfection was positive. In conclusion, the results demonstrate that: successful growing procedure of female chicken cell line was established, successful transfection of the RFP reporter gene and CRISPR Cas system into female chicken cell line was established, a strong fluorescence signal was generated in red excitation, and successful point integration of the RFP reporter gene into the Z chromosome was established.

What is claimed is:

1. A method for selecting a gender of an avian fertilized unhatched egg, the method comprising:
a. providing a transgenic male avian subject comprises at least one exogenous gene comprising a first nucleic acid sequence integrated into at least one position or location in both of said male avian gender chromosomes Z;
b. providing a transgenic female avian subject comprising at least one exogenous gene comprising a second nucleic acid sequence integrated into at least one position or location in gender chromosome Z;
wherein said first nucleic acid sequence or said second nucleic acid sequence is operably linked to an embryonal promoter;
such that a fertilized unhatched egg from said transgenic female avian subject and said transgenic male avian subject comprising the combination of said first nucleic acid sequence and said second nucleic acid, results in targeting at least one avian vital gene, thereby selecting a gender of the avian fertilized unhatched egg.

2. The method of claim 1, wherein said embryonal promoter initiates the expression of said first nucleic acid sequence or said second nucleic acid sequence on day 1 to day 5 post egg laying.

3. The method of claim 1, wherein any one of said first nucleic acid sequence and said second nucleic acid sequence is operably linked to an embryonal promoter.

4. The method of claim 1, wherein any one of:
a. said transgenic female avian further comprises a third nucleic acid sequence encoding at least one reporter gene encoding a protein product having an excitation wavelength ranging from 500 to 650 nm and an emission wavelength ranging from 550 to 650 nm, wherein said third nucleic acid is integrated into at least one position or location in gender chromosome W or Z;
b. said transgenic male avian further comprises a fourth nucleic acid sequence encoding at least one reporter gene encoding a protein product having an excitation wavelength ranging from 500 to 650 nm and an emission wavelength ranging from 550 to 650 nm, wherein said fourth nucleic acid is integrated into at least one position or location in both of gender chromosomes Z, and (a) and (b).

5. The method of claim 4, wherein said at least one reporter gene encodes a fluorescent protein.

6. The method of claim 4, wherein said at least one reporter gene encodes a red fluorescent protein (RFP).

7. The method of claim 4, further comprising a step of detecting at least one detectable signal of said protein product in said fertilized unhatched egg, wherein detection of said at least one detectable signal is indicative of the expression of said reporter gene in said fertilized egg, thereby determining that said fertilized unhatched egg comprises a male embryo.

8. The method of claim 7, wherein said step of detecting said at least one detectable signal is performed on day 2 post egg laying, at most.

9. The method of claim 8, wherein said detecting comprises subjecting said fertilized unhatched egg to a light source.

10. The method of claim 1, wherein any one of said first nucleic acid sequence and said second nucleic acid sequence encodes at least one guide RNA (gRNA) or at least one CRISPR associated protein.

11. The method of claim 10, wherein any one of said first nucleic acid sequence and said second nucleic acid sequence is integrated into said gender chromosome Z of any one of said female transgenic avian subject and said male avian subject by contacting or co-transfecting at least one cell of any one of said female transgenic avian subject and said male transgenic avian subject, with any one of: at least one Cas protein or at least one nucleic acid sequence encoding thereof, and at least one gRNA or at least one nucleic acid sequence encoding thereof, wherein said gRNA targets at least one protospacer within said at least one gender chromosome Z.

12. The method of claim 1, wherein any one of said first nucleic acid sequence and said second nucleic acid sequence is integrated into at least one site in locus 42172748-42177748 of said gender chromosome Z.

13. The method of claim 1, wherein said avian vital gene is crucial for at least one trait selected from a group consisting of: cell vitality, cell mitosis, cell metabolism, cell differentiation, DNA polymerization, RNA transcription, protein translation and housekeeping genes, and any combination thereof.

14. A kit comprising:
a. at least one first nucleic acid sequence encoding any one of at least one Cas protein and at least one gRNA;
b. a second nucleic acid sequence, wherein when said first nucleic acid sequence encodes at least one Cas protein said second nucleic acid encodes at least one gRNA, wherein when said first nucleic acid sequence encodes at least one gRNA said second nucleic acid encodes at least one Cas protein, and wherein the combination of said at least one Cas protein and said at least one gRNA targets at least one vital avian gene;
c. optionally a third nucleic acid sequence, a fourth nucleic acid sequence, or both, wherein any one of said third nucleic acid sequence and said fourth nucleic acid sequence encode at least one reporter gene having an excitation wavelength ranging from 500 to 650 nm and an emission wavelength ranging from 550 to 650 nm; and
d. instructions for the preparation of any one of: (i) at least one transgenic male avian subject comprising said first nucleic acid sequence and optionally said fourth nucleic acid integrated into at least one locus within both gender chromosomes Z of said male avian subject, (ii) at least one transgenic female avian subject comprising said second nucleic acid sequence and optionally said third nucleic acid, wherein said second nucleic acid is integrated into at least one locus within gender chromosome Z of said female avian subject and optionally said third nucleic acid is integrated into at least one locus within gender chromosome Z or W of said female avian subject, and (i) and (ii).

15. A transgenic male avian subject prepared according to the kit of claim 14.

16. A transgenic female avian subject prepared according to the kit of claim 14.

17. The transgenic female avian subject of claim 15, further comprising said fourth nucleic acid sequence.

18. The transgenic female avian subject of claim 16, further comprising said third nucleic acid sequence.

19. The kit of claim 14, further comprising instructions for breeding said at least one transgenic male avian subject with said at least one transgenic female avian subject so as to obtain a progeny.

20. The kit of claim 14, wherein said avian vital gene is crucial for at least one trait selected from a group consisting of: cell vitality, cell mitosis, cell metabolism, cell differentiation, DNA polymerization, RNA transcription, protein translation and housekeeping genes, and any combination thereof.


SEQUENCE LISTING (34 sequences)

SEQ ID NO: 1 - DNA, 3001 bp, Gallus gallus
cttggctcacccctgctcccggccgccccacagctggcgtatgtctggcattgccagcaccctgacaccgtgccgggctccccacgacacagcctggtgcccctctctataggggactctgtggtctccagctgcagccctgggcaggcagcatcgcaccttgcatcactggggctgtgctatgtgctgcccaggggaaacgcagctctcacacaatcactgcccccaaagtcctcagttcagtctttcctttgctcccagagcctaacagcttctcaaagcacatccaggtattttggctttccccacccgtaaggcagcagagaaggaagagctggcaggttcagatgcggcggtggtgtggctgacagccaaaccacaccccaggatctgtactggggaggcccttccaagaacagtgctctgagcacagccaccctccaggatgctcagctcagctcctgccagccttctgcagcccctgcatcatcctcagcactgcagcagctctctcccagagcccagggagcggggggacccaggctgagcagaggggcatggaagcacagagctcaggtaggccctgacacgctcagccccggccactctgctggtggagccaagcaaataatagtgccatggaggagtggcagttttcctctcagggatgggactttggggctgaacccagcgcttatgcccttcagatgggcctgaggtgcagatggggacatgggatccagtcaggccccatccccatcatgtgcaggtctgcagcccaccacatggctctacctgggctgcaccaactgaaactgtggcatccagcagtgatagctgtgtcactgactggcagtgaataaaacatcacctttgagcacacacacctaataccttttttctccaagggagtgcaagtcggaacacttggcaaagagcagagacaaatgtttacacatttctgccatcccagcttgctgccttcctactgacctggagagcatgcacagttcactccactaagctttttccagcaggatcccaccagcctggtgggttatagggcttgggctgccagcagggactgcagccggagaccacgtacagagagcctagggcatcctgcctgcagcatccctcctgccccaggcacacagctccagggatgtccgccccaaagggagaggacatgtgagccactgctgcacagtgacccccagaatagccaggcacctcgtacggccccagagatcgccccattgaaatgtacaggagacgtcgaggaaccccactgactcgctccacatcccccagctaaaaccatcactgccacaatgggccctgctcctacaaagggacagttcaaagtcaacgtctcccttcccctctgcctgtatatgaaagggagcaggaatgcgtttgaggccctggcgggcgggcggccagtcccgcttcctggggggatggagagaaagagcaggttggcaggaggagggggcccggcccaagaacaataaatcaaaggggaagggggttactgcccagccgaggcatcttctgaaaccccggggccgcagctggagcccgggaggagccggggctgatggccgcgattggaatcccacggcgacacttccgccgctgggatgcgatggcacagggcgggcggcagggcagcctttggggtgctgctgctgtggggtgcggtgctgcacctctctgcctgatgccgccccaggcctggctgacatcggcacctgtgtgcgtagccggggccgcggagcgaagggcccccaccaagcagctcccatcacaggcagctcacacagacgcggggttccgccgggactgctcacgtcccaccggcttttcgcatccttcccaggccgtcctgctggggctgccctccctcctcggagccacggggtcggggtgaaggcccaccgagggcagcaggacgacggaggaggcctcagcctccctcgccccaccacttttcccctatatgaccgccaatacaaggggcagtttcactcctccgctcacccctcccgctacaccgcggcgcacagcgacggctgcggaagcagcagcagtgccgtcctgccgagcctatctccttggcatatctcaggtgtgagactgcagaggccaaacagcggctcccggtaatccgggttaataaacaggagaaggtgcggtccaacgccccggcccgcttctggccgcggatcagagtgccggccccgagcagcgctggagaggagggaatgaaaggggaaacgctgcgacccgcggcctgacagctgcactgtgccggggcagcgcgacaggagagctctcgagagcaaatctcaagcgcgctccatcgcagggccgaccggcgcggctggaggacagcacagcagtacggcccgcgcaccgcagcactcctctgcggggagagggagaggagcgcagcgctgcggggccgggccgtgcccggggagcgccgtgcgggacactgtgcgccgtcccggggccgcgccggtgggcggacggagcagagcgctaaggccgtgcagacacagacggcaccactgttcccaggcgtcggggcggctcccgtgcgcccgtgccatccccgtgcaatgccccgtcgccgccccgggcgggagatgctgcgtgcagacctccgctggcgggcaggacggcgcccccccttccttcgcccaacggccccgggggcagcgcgacgcgaacccgaccccccaccccccagagaaaggggatcaagttcacgcccgacccaccggcggggccgcgccgccggggccaatggggcgggggcccatttccatacggtccggcccggcccggcccggccccgcggcgctcagtggccccggcggcgcggtagcatggaggcagctctcggcgctccgcgcccggcttgagcgcggcggccggaacgagccgcagccccgtcggcgccgcatggagccctaggccgcggccacca

SEQ ID NO: 2 - DNA, 3001 bp, Gallus gallus
gtcgtgtgcggaacttcccggggaacttcggctctgcggagggagaggggggagaggaaatttctctgtacgttttcgtcttcggcatttttttttttaacgtaatgaaattaaaacttttggagctcagagttgacattttgcggaaaattgagttatcaaggcagtaattatttcacagggagataaaactctcatagccctaactgtcaaatagggcccttttcagattttaattacaaaataaaattagtctgctcttcctcggaagggtttgtgagtggctaaacagagctttccccaatactggtggtcgtcaaactctgctaattagcaatgctgagaaattccagttaacaaggacattctctaagtctctgcaggttccctgccgttcgccttcatttccataagaagattaagagtggaggggaacacactcaaatgcagatgcagaaaagaagcgtttttaacaagcatcataatagtaagatgcttggctagttctcacctaattactgcaagttaaacctctatttgctgctaaggacaaaaaatggagctgcaatcttccatctccacaagaccgcttaatagtttgcttagattggttactggcaggttcggaaaaataatatgaaaatgcgtatattctttccgatacagttaacccgataggctgtatcctcggggctcagagagaatcggcgattattagcacaataaatacgcaaaatccaagtcgttaacgttcattttcttttaaagaaacgcataacctggaaataagagtgcgtgtgccgcagagcccatcgccgaggacgtttgcggactatttgcttaatgcgaccgctttgcatttccataaattacaccgaagggagcgggtttataaatccgctcggaccgaagcatccccgcgcggctcccccagcgccgtgcggccgtgcgcggagccgcggggtccggccgtgttcctccgcgcacccaccgcccccagcgcaggaggcggctcctcggggagccggacggccctcacctcgttttctagagcgcgttacgatgtagatacgccagatacagttgattgcgtcgataattaaagcagttagttcaaacgaagctgtaaataccgcccgataacgcactcgctgggcaattcccaccctttatgggtgcgataaacgcggtggggaaggggcccggccccgcgcagagccgcgctccggtatccgtttgttacagcaccgccgcccactcctccccgcttttcaaacttaattatccccgagattgcattattatcattatgatttggaatgttgttatcaaccgcgggccccgggcacgaccaataagagagctttcagcggccgtgggagcgcgggggcagtcgggtcgcgcagaaatcgcgcatcaccgcggtgtgcgcatcccgcaacgcgcgcccgttttccgacgctttcaacccgtcacatctctccaggttatatatacaccttcgaattacacgcgttaaagccgatagcgtttatttgcataatccagagatgcaccctcgcccgcctcactttttctgcccgttacccggacgggggggggcacggccgctcccttccccccgccccggctcccgccgctccgcaccgtgccggcagggggcgccgcagtgccgcggctgagccctcgcggggcgcccgggcgctgccccggccccaacccgcccctgcgcggggccgcgcttctcggggggcccttctcggggggcctcggcgctgccggggagcgcggccgcgggcaaaacaacgccgtcggggcgcggcagcacgtagagcggggccgggaagcgcccccccccccccccccctttctctcccctccttcctttgtcgtcaagtgcgttttagcaacaaagatcccaacaagagagtggaagtaaacttagccggggctttgtgcggccccgtgtagcgacaacaagagaaacaaaactacctatttgttcctgcgggccgcccccccgcccggcgcgcccacccagcgtcgcacagggggcgcgtcccggggctgcaggccgcgcaccgcgttctgcggggagggccgtcgggggacctccgctcccgcttccagcgggcacctccgggttggaaggaaggagagagaagggcacggcccggccccgttctgcgcggggcccgctcggctcctttcggcggggcggccccgggtcgggggctccgctccccgcggacggggcgagcgctgcgcacgcagccggacggctcaggggacacttcgcaacggggcgcggggtcgaggggaagccgttcccgctccccctatggcttctcccgacatcaacttaaaaaacaacaacaacaaaaaaccaacaacgtagcaacaatgctgtttacccacttcctccaaaaggcgtgtgtgacctgttgctggaaggggagatacaaaggcttcccagtggctgcctgggccggccctgggcccgccccccccgccgcgccgtcccggcggctgagagcccccgccccgtttcatgcaaaaccaggggtagagacttgtcctcagtggcggagccgccgagcgctgattggccggggcgaacgcatttattccctgacaagcccccatcacatggatggttgtctattaacttgttcaaaaaagtatcaggagttgtcaaggcagagagagagagtgtgtgtgtttgcaaaagggggtaaagtagtttgctggctctttaagaccgaggtggagagagagagagagagggagagagagagggcggcaggcagagaaaagggaaaaaggaggaagaagttttttaaatccgaggctccggcggtaataatagcagttattagcattattaatccatcagcgaagaggaagattctgatctcgatccaagtgtggttttggggagtccccctcccccctttcatacccctccctaggaaaaaaaaaaaaaaaaaaagtccggctt

SEQ ID NO: 3 - DNA, 3001 bp, Gallus gallus
aacaacaagcagtgcatgaaatggagacaacgcagcttaaaccctattgttaaaagccccagccatctcctgctagctactactcaataggcttcaaactgaaaggcacagccttgtgcacaaagaaacctcatcagctcttaattttcctacagcacggttactaagattaatcttaatgagcaaggcaaagttcttagcacagctgtcattccataaaaaggaacagcagcaaaacacaagcagtgagtttaaggaaatgtattacacatactttgcctcgctccttgcaaatctcacagtgaggagaacagcaaagagacagtgagactggctgggtgtgtttggaaagcaccacacggaatctcagttcacagtgggtttaaatactgcaccctcactgacagagctgcacacccagctacaggactggggaagtgcagctctgccccaaacactgaggggaaaaaaataaagagcctcgataaagaacttgccatttaaatttccacttgtggatacggcgttgttctgcttctggttatcatatgcgggaccaatattggcaagatcctgaaaaaggaaagaaacaaagctaaacagatgcacagattcaggcaaacttaccacaaagccccagcagcagtcctccagcacaccacttggcacaacactggctcataggttcatgttatcttggatactttatgcttatttcactgagcataagataccagcaaaaccagcctcccatccccctcccccccaatcagtgtgctctcctttcatctcccatcacacacttggtccagaagtccaaacttcgggtagtcttcttcgggatccttactccctgggtctggatgctccttcactaagaacacatctctttctttacactgaaacagagaacagaacaagatcataccaaatttgttatcaattgctccccattttgctctccttgagtgtcagacagcagttatcactcacagaaaaccctccatgaaccccaactacgtaccatagttttgacaatattattaggccaagtcatcttccctggtctctgcctcgtcgtcatacactcccagtatttatcaatgaatgggataatatcctgttaaaaaaaccacaaatccactcttaacaactgagtctgaaagccacactgccaacatttggctgaaattgctcttcagtgaaaccacagctggctttaacacgtgatgactgttatgacagccgtaccttatctttggagaacatggtttttggatgctcatcttgtgtccttgactgccacgttaagttggccagagcactaaggcacatttccttgagatcttcaaaacaaagataggccaaagctcaagctgcattccatggggaagatcagcacacaaccacagtgatcaccaaacacacagcaccacaccaagcccagccctatcccatcatcccacaccaaacacagcatttccacaccttatgaagtcagcagtaaagcagcaatgtttcagacaaagctgcccattcctgctgctaacttctttggtcatttctgcaagtgtcagcagaaccctctcactgacatcaggtaacattaccttgtgcaaggtttccatgagggagctcttacacttacttgcttgttttcttaagaaatatgtgttcccactgtggtggcaaacgttgcaatggaaactgtagttggtcatgaaaggcaaacaagatctggaaaagagcagactgatttgtaagttctgctgtacgccttacgtgctgcctcaggtaagaaatgaatgctctttccctacataaagaacaccttattgggtcaatgataactgcaccagattaagtttatcactggaaaactgtgaacttctttcctcccacacctctttacctaacaaaggaatttcactgagcagcacacacagtgctccaattagtgctgcacagaaagttcaggactgcacacagcaccatgctcagattgaccaagcacattaataacattaaaacttccattttcctcaggttttagtgaatgtctggatgtcacctgccttgagtaacctgtgagctctgacaggaggggggcaaaggcagcaccaactgaaatctccacccgaggtacgcagggaggtggtttggtgttgaaagaatgggggggatggggggttacaaacaccaaaggggcactgagagcatcaagggccaccaatggggcacccgagggcacccaaggggccacgaaggcagcacctgagggccaccaacccccgagggccaccaaggggcacccaaaggccaccaatggggcaccccacggccacccaagctccacgcccggcgctgcacacatacgtggtgtcgatgccgaaggtgtccgccgtgaaccacttggtgcagatgccgcactgcagttcgatctctcccagctgccgcccgttctcctcatccaccgaccccgcctgcgtgtccatggcctcggggccgtccggggcggcctcctgcgggcagcacagcgccggttatcgccgtcagagccctcatcccccccccgctatgtgccccacgtaccatcggatccttcccgctcgcggtttcggtctccacagcagcagcggcggcgtcccccggcgcggcctcccccctgcaaggccacaaacgacgcctcagcgcggctcacccccgagccgcggcctcccagccccgcgctcggcccccgccctacccgctctcggcgtcggcggcgctcggttcgggcggggcgggcggttcagcggccgggggctccggggccgcctctgcgctctcccccgcgggagccgccattgccgctgccgccgctgccgccgccgccattgctgttgacgctcccgcgagagttggccccgagcgccgctctcgcgagattcccggcttcaaagggcaatgctcacggtgggacccacgtcccgatggcagcagcgccccctaccggcggccg

SEQ ID NO: 4 - DNA, 3004 bp, Gallus gallus
cacatcacaccaaattcctgcgctgttttgcatctgcttaccctgcccttgtcctgcagcagtgcagcgaagcaaagtgcgtggccctgggatgctctgagcatcatcacccagcccccatggccccacaccccccagcccccagtgggcaccccagagcccggcctgttcccatgtgggaagaaagggaaggagcagaatgagggagcgcagcaaggctcgcggctttgatctaatgagcagactctgcccttcggtttgtttaatcgtttcacacttgctctggaaataatttacagcaggaagcagtgggataacaccatggcaagggctggtttgagcacagcgctggcgtgggggtgccctgtgatggagggcacaggtggggcagcacctctccatcagcccagcaccgaccccaccgcctcttcgtctcccagcgcttggagtcccacacgtccccacacacaaataggagtttcaagcctcagttttggttgctcaagtcacagcctggttccaatttgctgccactgccgagatgcgatgggaactgtgcacactgcgtggggtgaccccgtggagggagcggttctggcagctcagggggctgctcccaggctcaggatacaactgggttttgggtgggacatgcccatgctcccatcacgcaggacacagggcagtgctgggggctggaggtgggggcagcgggacccgcaccacgtgcaggagctgcacacatctggagcttgcagggctgcatggcagggttggctgctcgcctccccatgtgcatttgtttgtttgtttgttgctgcagcacatgggccgggctctgccccgaattcaggagctcacagcgcaacacagcagcatcccgcctggcctacaggtgggaaatgtctcagcagacccatacacacctgcactgggtcttagatgcgctgcactgtggctgctgtgacccatggggttgggggggctgcactggagctgcaggaggttgtataaaggcctgggatggggctgcactgggactgggggggagaaggggggttgcactgggggtatggagggactgcatggggggagttacatggggggctgcatagaggtgtcgggggctgtttggagactccgaagctgtttgggggctatgcaagagttacacggggggctgtatagggggtccagggctgcctgggaggggtacacggaggggctccataagggtgtcaggagctgcaccgggactacccagaggctgcagagcggcgctcggagctgcaacgagccgcggaggtctgcgcagggctatccgggaccgcaccggagccgcggggacgaccgcaccggagcgtccggagctgctttgtggccgtgggggggtgggctcccagcgctgccccccgcgccgtgcagggaggggcgtcccgctccgtcccatcccgcgccgctccgcgccgtcccgccccgcgccgtcccgccccccctccgccctccgcccggcccggcccggcccgcgggggctgagcggccgcgggaggcgccggcgctcggagtcgctgcgctcggcgttggggcgcggtgtgttcggggctcccggcgcgcgccgcgcggctccggttccgttcggttccgctcggttcgagccggctcggccggcggcctccggatctatgcggctgtgagcggcgggagcagagagggatgaagcggctcttggccaccgggctcctgctggccctgctgcccgccccgacgcgaggtgagggccccggcccggctccggcaggaagtttgccggcagcgcggcgcggagcggcgggacgaggcggtgccccgctctgaggcggcggggggtccgcgggaagccgagtgcggggcggccgggagggtccggaactcggagtgcggcggggggagcgggctgagaaacggcgacggaacttcggagttcggcaccgcggggttcgcgcggccggaaccgagaagggggggtgggggggggggggagagcggggacggtgcttcggggcctccgcgtggagcggagccgtcggggtgtggagagcggcgctgcgggcgggcagagctcggggtcgcggcagcggggcgcggggccgccgggggtcccctgtgctcggaaccgagggctgtgggggtcccggagctcgggacggggctcggctgcacggaggtgcgcgcggcggccgcgctcgtgctgttgttccgcgagagttttttgaccgttttgcaaatttctctcggtttcctgtttcttgaaagtcagccgggtgcccctcggcccccctccttaacaccccccccccgccccccccccaccccaaacctcctccccggcatcggccccggggcggatccgtgtgcagagccgcaaagtgcattgctgccgtcgggggctggagggggggggggggtggggaggaggggggcagtggggccgccccgacgggccgggccgagctggagcctctcgggacgctgtttgtagcggcagcgcgcggccctctccccgctccgtcccccggggctgacggggacgctccgctgccgtcggggggcgatgggttcgggctgcggctctgcgacggctgccttcgctcagtgtgaaactgcttcggctgccccgagagcggcagaagcggttaccgtgaagcgagagcggggacagagaaggggaagggtgagccggggtggaaatcggaagcgattctggtacttttcagcgagttgctgtggtaactgagcagaatccggctccgagggccagacattgtaacctttactcatagcgagttgtgccgagccactcatttcgggactgagctcgtggcagcccacgggcccgcaggcggatggtttttcctaagtggtgtttgtcactcaggtgcccgacccgctgcttctccccgtccaccctcctgcttcggcgggcacagcccacccccaggaccaccccaggtcgcccctctcctta

SEQ ID NO: 5 - DNA, 2952 bp, Gallus gallus
cccagcccctgtaaactggtctgtctgtatcacttcagtgtaagtttgagctccatccctctgaaagcggtgaccctacttacagtagtaagtgaaccctcgtagaaggaacttgcatgaaaacaccttccagcccttgtccaatctcacctgtgtttcagccaagagagcttttacacatcagcttctaccctggaaggactatggaaaaggctccttgggacatacttctgtcaagtgactgcttacattcccttccataggcttaatgacccagctgatgcttacacagtgactaatgctccataatattaggcacataatgaagtgataactgcaaaagcagggagtggccacagcattaagagtgaggtgacacagccttcagcttaaaaacaaatcaatttcaatctgttgggaagaataggaccaaaacagatctgaattgcattgctgctccacctgaaaacagcagcaaaaaggaatatgagcgaatatttggatctgctctcagagtcattctcagcaggtgtttttaaaaaatcatcatctctaaatattaatgggcagtttatccactttgcagcttaatggtgtcacagggcatatgtcccagatccagcacctcaggaagcctcttgaatgtctacatcctgcttcctatagcattctctgggcctgtatgacttatggatttaccttctcaatatgttattatgtgtaaaatttattttagggaaaataactgaatttcttttcataaattctgactttagcgctagacctgattttatacactcacacaagccttatttctcctataaagacatttcaaaagataagtgataatttggtaagagtcctgactgaaagcaaactgatgtcactagagggcgacacgtggaacaaaaaaacagtaaatataaagcaaattagagacaagtgagatgagaaaatcaaaagataaacaaagattgtgggaaaattaaggggaaaaaatgaaatcaattccatgcagacagcaaagcaggtccatctctgggagggaaacagctcaacagcaaaaaaattataaaccaagggagactggggatttgatggcagatgaggtgttagaggctgtaaaatgctaatccttctgctatgggtgcccctagacaagctgtgtcacctctgatgactcagtctgcagcttgtgggatggcaagagtgcatcgtggaacttctcagcatcgtgtggcatggcctccagtcttctgtctgtgtcaatagtatttactatttcacttaatgcgcactgtagaggcaaacaaatggagccccttaagttattattgccaagagacaaacaagtgctttggtttgcaaacacaacttccctgacattgaaatcctgtgtttttccctctctttgcaataggctgtttttctatgagctcagacattgtttcacaggtagcacacatatctccagggaagccctgtcagcaaagggactgtgcagatcagtggccaccacaaccatatcggttacatggcaattttgcacatttgacagataaaccctttctctgtgtgcattggctaagcccataacagcaagcaagcaaccgtgtgtgaaaccaacagccgaacatgtggggacatggaagagcctacgggaaacggaacgcacagcttacaccacgtgcttcagggcaagacagcatctgcctccacattgtacccctcatcgaattataataacttgctcctgcatccctaatcacctctctacctggaccctacccagattctgagaaactaaaacaccataaattataaagtcataaaaaatgagctcaatgagggcccctccaagttgcaaaagcaccttcaggtcagcaactccctgccactacctagcaactctggagaaagggggcttaacgtaacacatgggggtgtctgctcatcatgactaagctggggagggagaaatatatggaagtaaggggggcctatttctgttgcctctagaagattattagcagctaatatctgttcctcatttcttttcagacagggaggagcctgtgtcctgtcctttcactaggtgaagtagggaatttgcttgatttgtcatatgctctcatttgtggatctctcaggttgttttctgcattttataaccccttttttgtaaactcatgcttatttcaaatgtcatgaaaagaattgacaaatatctgaggaaagatgctgagaagtgacacttctttgtgctcctccaaaatagatgtctgtctcatccttgggttagcatttagaaaacctggggagctcgtctttgagtttgcttccctgtgatgcagacaccatcctagaataccgccctccccgccggccccagtctgtattcccttgcaaggaggaatgcttatctgtttacaatgatggctgcagtctgcaatgcagacacgaaaagcctccgcatgtcccgggggggtctctgggctccccctccattctttgtacttgggtggggaccgatgaggaggcgtaggtgggtagcaagactgttaaatgtgagggcgggggtgccagcccagcccgccgcacgggacggccttgctcacacctgctaggcctgaggtggaaacaaggccccctcctcccagccccggggggtcgggtgtgcgggcagcaaagaactgactcccaccgaggaaccaaagggggaagctggccttgctgtcctagtcctcccccactctccgaatatccccatagccactcccctccaatcaactgacttttaacatgataatggtcgtgacaatctcttggctaaagccagtctggggaggacataaaacattgcccctcaccgcctaatggggtatctccgtgcagtaggcgcgtatgcaaccagctcaccgccagcaacggctggaggtgtcgtcccgacca

SEQ ID NO: 6 - DNA, 3002 bp, Gallus gallus
ccccccttttttttttcttttttctttctaggggaggctttcaaatgtgttcatttatttccagtgtttacatttatgtgacacctgaagagcaacacgcattgatttgtcacacagctcttggacacacctacccctctgcgacggcagtcagctaggaacaccgctgctttgtgtgctttggaatcggagcacatgataagaataaaagcatctgtgtcaatgcacacgcttttacctactggcttgctggaggaaacgctgccttcgtggctttcccctaagaaagcatagaaatatcccgttagcaagatcgagcttagggaatcatagacacctaggatgttctatttaaaattacacgtttcaaaggaattactaggtcattataggctgtaattccagtgtagttacatacggtctaaaccttattctcctctccacattaagtaacagacccgaactgctctctgattgttactttggggctacaaggagctttggggcatcattaaaaatatttccagaaagaatcggtagaccacatctgacgtcccaccctctgctagaatcagaccacatcaaagggaccctttttgttttccgaaatggtcaccagacaacttaaaatatacaagggtaggcttatgttttattgtatagatgtctgcctagaggagaacgacgtcaggttgctggctattcagcgctgtgccgagtgagacgaggtagctgcagccctttgatattttggtggaggtcgtattcatttcatttgaaagggacccgttaatgacagacctgtgagcgctcccctccctgagaagcggagcgcgcaccgcttgggaagcccagggcggaaagatttgtagatgatggtgctaattatgaaaatgtgatcaagatggaagcagtttgagcagaagtcattaacgcgttacccttccacccactgctcttttggctttattactgcggtgggaggaggattttgtgggcttgtttttttcagagcgctgatgaaaaacgccctgcaaatccaaaatgatgcgacgtcatccgtgcagctccaggttggagagatgctgtcatcaccgagtagtcgatacgcgcagtgccgctgagcgcatctcctcgagccccggggtcccttggggggtcttcagctgtcctacggcccagcgaggaccgcgggatccccggggggcggagggacccggcccggtgcagccatcccccccacaggctgtgggagtgggggatggagtggctgcgttcttattttcaaacggccacggcgccctattttcattttattctcatttctatttacttttcctaaataactggaacagcgttagtgcctggaagtgatactgcgggaaaacaaaacagcaataaagaattaattgtacaaacatttctcatttcccagatccgactctccccgtgaggcgggggctgaacgggtggggaagaaaaggaggaaggggcggcaatggcggagaggggagcttatgggtttttatgtgtttccttttatttcagacggccgtgtaatgaaaagccttttagccttggagaactactaaatactgagttcagcttaaagcagggaggcaggtcgcatcccttcgcttattttcccgctagtaaaagcttccttttattaacgtgtaagaaaaccctcttgctctgcgtaggaatgtcctttaaaatattttatagacctcttcttgtgcgccgacagagggaaaaaattatatcggtgaaggtcaggcagttgaattctgtcgtgcttcacttcaaaggtgtatttgcgtgccctccgagcacattaatactaattatcgcccaaacacttccccaaaactcccactgcccatttcagatgcaaatctaattaacgagccgcctttgagcggcacgcaggaccgccgcaacggagcgagctgttggacctcttgccttcccctccatttattccgaggcgtgggcagcggtgtgaagctgcgcggtagcgagagcggagctgctgcagctaatgaggcaccggagttaggaaaaaggattagccccaacctcttgcaaatgcattgagcacccatgtaacaacatttgctttccggctgggccccactttgtcgaagttaatttagaagtgactgcgtcttgaaagacctacctaaataacgcgtgttattcgtaactgggagtcggtggatttagggatagctgtttgctttcaacaccaaagcgaggtgggggggagtccccgagtgaaagccaccgggacgctctccccccggctttctcatgctcagcgtttccgaaagccgcctaaccgaggtgttcgttgagggcgccggtggccgcggggctgcgcggggggcggcggaggctgcgcggcttttgcgcggagcgtagggcggcggcggcggccgcgggcagcccccgcgccggggggggaggtgtgcgtgggggcagcggggatgctgtgtaattttatccacgctgcactctgggagttcacacgagagcgataccttttgcctcctctgcttcgtatcaattggtcgcagatcgggctattgttgcggggagccgcttttatttctatgctccgccggtcagctatttccccgcagcctcctgccgaaattcagcggggctctttctcccccctcctcctgcttctcgtgccgtctgtctagctggaaacccaaggagaaggctgagcgcggcagctccatctcacgcaaacagctgggactcctcggcggccttcgccgatgtctattgatttaagtaaatctgagacagataaaagggccagatcggggccgatgataaaaacttgccgatgcttcgcctccggacgcggtgcgcgccgtgccgccccgccgcgccgctagccggacgccgttaatgggagccccggtgccgctcgccccgagcctccactcccccgcgggctgagcgcggcggccaccatgccacga

SEQ ID NO: 7 - DNA, 3001 bp, Gallus gallus
cagggctcacctccaagccctccctgtgtgtctacccaatggggccacgccactgtggtcggtcctgcacagaatgggcacagccccatgctgctgttctcccgtcccgacattcccaccgtgttcagccctggtgccactaactgaacagagaggagagtcaatggggagcagctgtacccccctccctgttcctggagctgtgggtagtgcagacgaggtcattcccacgtaacacagtgcggtggggtctggcccttggggggatgctgagttaatggtgagcggtgggatcgccccgggctgctcagcaccactcagccatggaggagagctgctgcctccacaacgccccattaatgctgcaggcagggctggggagggtgtgagcaggggagtggtgtccctttggctccactgggggggtctccatatgcctgtttggggatgtggggctgagcatccaggcccacagacggaagctcctctcactctcagatgggtgctggagtgcaccttgcaggtgctgatctccagcggttggggttggtgctgtgatccctgtcccagcagtgctgggggctcatggggaactgcagccacaccttctctggcagaagggcccctctgggctgcgggtagggatagtgctggagcaggaccttgctgagctgtggaggctgtcagggatatggcatgatggagcaaacagaggaacgggacggcccctctgctgtctgctctgtggctcagcccagcttgggcaaccctgggatctcctccagccaccagcagccgtgggctgccttcctgggcattccccattggccatggcaaggcgagggggggggagttgggggataaaatcctgacctggatatccccagctctacggaaaaggacgaagggagcagagctctctgtgggtgtttgatgcttcacccacagcattcggtgttgagaccaagggggccgtgctggctgcctcccccaggggtgacagctgtggcagcaggaacacagcaggtccgtggccctttgccgggatgggttttctttccctgccccacggcagctcagcccctcagtgcctcaggctgagctttctctgcccagcaggagagccagacacgagcccagcgcccagttccccgtcccgtggcatccctcacccgcacaaaggtggggaaggacgggggcggcggcgggagcactgtttgtcattgtgtcagcgacgtgtggccgcccgtgggtgatccccacgtcacagccgcgctggcactcgagcctgtttgctgagggcagggggcacacggagccgtcagcccccggctgcctctgtccccccccggcgcaccgtcagtcggacgcggtgcagttgtgcccgctctgtgcgctgcacggtgctgcacggcccgacccggcgcccacggggggggtgaccacgagtggcacctgggagagaggcgctgcatccagacccctttcctttcgatgcgtaagaaagacgggtattgctccagcgtagggcggtgggagcacctcgtgctgcccgctctggtgctcggcttgtgggcacggagctgcgtcgcagtcgggattggtgcagagggcttttctctgagctgcacccgagggctgagcacccccagcgcccgccctgcagccccccgagtgaacgtgggctcaacctggggcttctcgagacgtttgggagggcctttcaggaacacctcccggtcttctgcactgagaagaaggggatggcaacgcgcggtgcccgctccagtgagccccagggggtggtggtggtggtgggcagcgtttgggggcaaaacattggagggtgcctgaggaagggggcaatgagcgagccgggccgctgtgtgcgtgcagggctgtgcggggctgcggggtgcgtgtgtgtgtgtgcgtgtgtgtgtgtgtgtgtgcgtgtgtgtgtgccggtgcgggagggccgcgcaggagcgctgtgtgtgcgctgtcactcagaggccggctgacaggagctgcctttccagcggcggcagatagcgctgattgctgattcgaggaaacaaaatagcaattgtaattatgggctctgataagataaaggagggcgcggggagcgcggggaccggcagcggggcacggccggcaaattagcggctgtcactcagcgcggagcaggcggccccggccaggtaccggccccggggtcccgcccgtccgccgcagccccgccgcagcccccgccccggtgcgcaacaggtggcggggccgggagcagcggggctcgccgaacaatgcagccctcggcggccctacccgggctgcggggctgagccccgtcccgccgcggccccatggccccgcgcagccggtgctgacggacggccgcccctccaggtaagcgcccggcggggacgtccggggaaggggcgggggggtctcgccgcccagtacccccggctgcgctgtttgctcagcctctcgaaactcctgcccacccctccgccccggtaatccttttaatgtctggcacgtccagcgagagggctcggccccaatccacaacgcggacccttaagcctcgttttacagatggggaaactgaggcacggcgcggggacggcatccctccgccggccgagcccgtagtgccgccacccccgaggggccgccgccggaggtccccccgctctgccgccccgccggggccgccccccgggctctccgcccccgccgcctcccgggccggcagcggagcggagcggggcggggggctccccgcggcgggggggctgccggccgcgctcccccgggggctgggcgggcgggccggcgggggcctttccccttctgtagccgcttgtgccctcctttcctcggggcgaggcagggcgagaggagtcgaggagccgagcagcgccgcgccccccccccccccccaccccaccgccgggctccgggctgccggccccgcagcgctccgcga

SEQ ID NO: 8 - DNA, 3001 bp, Gallus gallus
cccaagggagggctcaggaaggggcattaatagcaatcccagtaccccgctgcttcttcagagatgttgccacagaggaatgtgggcttctcacacatacatctgcctgtatcttccgatatatgccaaatctgtcaccaaccaatgagcatcatcttctaattagataacagttggtcatttttcaagctttctctattttcccatgactttttcactggctctgggaaaaatcagcctaatgcacacaaaaagcggcaaacaagtaaacaaacaagaatcactcctatttgctatatgcaaatgacgttatcggcagtgtctttggaaaggatgctctttgtggagtcctcgtttgctctggtcgtgctgtagaaagcacttttgtaattcactgttcaagcttggggaacagcggaattcaaaagcagggcaaacccattaaagcaatggtgaatgggacaaaaccattaaaggaaaaaacacagagcccttgcttttcagagaacagctcaaagcattttgcttcttgaagaaagtcagcaatccatggattgcttgaaagatgaaaggctgcttcgtgctgctgcgtcctcctggccttgctcatctagggtagtaactcaggtaccagcaggaaaaggtgggctttcaaaaagcaggtgggtgcttcctttcatgatcctattggattctaccagcaactaggcgcagtgctgagtgtgatggaattgctgcttttcaattccacaatgaagagtggaaagttcacagtggactcacccagctctctgttcacccatggtaccccagagccagggctagcagctcctggccctgtggtttgtctgctgcagtgcactaaggagataaccaagtgcaacctgagatgccaggagaccatctccatagagtcctaggatcatgtgagttggaagggacctgtaaaggccatctagtccaaatccccagcagtgaacagggacacctacagctagatcaggtgctcagattcccatctggcctgaccttgtgtgtctccacggacggggcatccaccagctctccgctcaaactgtgtcagtgccttaccacttttattataaataacttcttccttttatccaatctaaatctaccttcttttagttcgaaaccatttccccttgtcctgtcaccacagatcctgctagagtctgtccttttttcttttatagcccccttttagatgctgaatcacatcaccctggagccttctctttcccaggctgaacagccccatctctctcagcctgcccattggaagtgaggtgttcctttctttccatcctgttgcctgccctatcatggtatgtgctgaagtctgcagcccgtggctcctcctgcagccaaacagaggggatatgctgtgcatcacggctcctgtattccagactcgggcttttgatgaacagcttggtctttgtcccaggaacaatcagcacaactccttcttcttgttgtttaatgagggatgccatccccttgcacctgtctctccagaagtccctgctctttcaccacctcgacttttcctcctcttgtcaggttcacaccccgaggtgatccatccagcaccactcagcccagcctccaagcttttgccatgctcagccccattaaaaacctgtttcactccgctcaacattggcagtgaactaccacttgccgctgatgcaagcagtcagtcacaggcagcgactgctccccaggcaggcgtgcggcaccactggcaaggcaaaggcagccccgggacgtgcagatgccaacacacatgacgcggcaagcactgcagcgggatgcttccttctgcccaggcacagcagggtctggaaaattgtcactgcgtgtcctgaggcgagggggacacaaaccccgagttaagcaatccctgatgtcaacttcgaacagcgcggaaacgcgctgtgttctaacttggaagtttcagacggaattgggagagtttggagaaaaaaggagggctggagaagcaggctgggcagagacagtactgttaaaggaaatctgtcttagggaaacaacgtacaggtcattttgtgggctggggaagcactgcctgtgagcagctgatgatttaatgcccgtaattttcagagaaggccctgagcatcctcccagcacagccccccgcctcccctcactcatccttttggcccgctagttccgggcccgtcttctgccaggcgctggactttgtgcagaaaaaatacccccatctcccagtgccagcattattaataattaacatttatgacgctggaaactggaaaaaccagttggctaaaacaaagccgggcacttcgttcttatttttaaggctaacactgaatcatcccgaagcactcggctcgcgtgtgccaacgccgcggggccgggacttcccccgcgggcccgagcagccccggggcggctgacgaacagcggggccggagggacggggctgccgacggggatgggccgggaccggctcgccactgccccctgcaggaagcggggttgtaaaggaagggggaaaagcagaccgagaaggcgaagagggtaacgaaggcaccgccgctgagacggctttagcgttatcgccgcgactccgctctgcggcggttcttgtactttatgcccggtcgcgtcatatcgcgccgcgtgacgtcatatcgcgccggcccacgcccggccccaccccgcgcccccccccggccccgagcgcacgcgggcattcccgcacgcccccccgccccgaggcctcgctgccgtcagccctccgcctgcgtcacgggggcggggcggggcgcggggcggggcgagagcgggctcgtctcccccctgcctccgcccccctccgcgccgcgcgtgccgccccccccacgtgtcagtttgttttctccgtcgccatggcgacggcgggcggagcgcgcatgaggtgaggttataaaaagggcggccg

SEQ ID NO: 9 - DNA, 3001 bp, Gallus gallus
ttggacagcagagcagaaaggtggaagcaggtgactaatgaaatgtaagatgagaaagaagtcaaaacaaagcgtgtttttatttaaaaaaatatctgttggaattaataaggaagtggcattcattattttaggaagataacctttttttccatcaggcctatcacaccattttgtataaagggaaatgcaaaatggcagtctagtttgcctttgaagaagcgaacacttctgtaagctacagatgaagtacacagcagcagactatcgcttcatgagtcccacattcacttgagcagaaagtcaccctgctacattcctcgttgtggtggtgctgtgttacagagccacgagtggcatttgcaaggcctgtcaggagctcctatggtacctggtggctcctaccgtggtgcccggtggggagtaagaataggaaaaatgattcattgaggtaaaagtatcacactggctctcttttgacaaccaaaggtaaaatgagaaaccattttttcccctggggaggtggattctaatgggaggtcaaaaataggtggctactgatttgccaggaagaaaattcatttccatttctctagaaatttgatgtgagaaaacaaactgggtatttgctggctcaaaatcagcttctgggtgtgctaaaaagctcctgaaatcattaacagaaatctttgtgaggcttaaagcctgttgatatcaggcgtccctcacctcgtcattatgaaacatcccacccgcgtccttccactcctcccctgtctctacaggaggtacaggtccccacaaatgctggcccttctcctcctcacaaggacagagggacacaggcgtgcccctacaggcacacctcatgtagctgagaactcattaaaagtggagcccacctccatccctgctggcaaccccggctcctctgccccggctgctctctcacaccaggcagcagtgggctccaccactgccaggctccgaccgccccaatgaggacttgacatttgttcttgttttctcctgtcttccctaatgggtacagcactactaagcaaagaggaatgcaaagaacaagggagttcaaagcaatcgagcctacatttaccaagaatgtctggatctttgcccgcccagaacctggagcatttacatagtttcagaaagtttaacccttttccattaaactttccagcacagggcacggtgcttctctcgtcagtgcctgagcacactggaaggcatccttcacatacccacctctaggcgatcccactccaccttttatttcccatttttttttcactctgtgggacagtgatctgagctctcgagtcctctgttgtgagatgttcatagaactgcatggcaggggaggggtctcttacaatagctcccaaacactcggctgagattctcgcttacacaacatatccgagctgggtttgaaagaccctacttgggttttctactacaaatttgtctggtgaagtgtaaactcacttgtaactctgtctgcaaaatcagtgaagaaacacagatattctgaaataattacaacagcaagcttgtattttttttttctaatgatgaactctgagcaaaccattggttccatttaggaattatatcagttggccgtttatttctgactttcgcactataaagaaaatttatttttgaaagaggtataaactctagtaatagagagggctccaaaaacaggctattttgccccaacccaaggctacgtagtgcagcggcgctccgtagctcaatcagcaaacctgagcaggaagtcagaagtgctctgactgcccattacgtactgtcactaattgttagtgacaacagaaatgctaaaataaaaattgactcttcacaattgcctgctctttagtaatcttccaccgagtgccgcttgagaaaaggccgtatgagcgggctaccgtaggagcgggctgccgtgagcacagcaggagacggtggggctggatttgtttacctgcaggtcagccctaactatggccttttagaaaaattacaggcactgagagctgcttctggagatgtgcatacacatttgcagtaccaccgtgatggagtggcacagcgttcacccgagcaggccctgcgtaaacagacacatgcagcacaaaaggaaacaaaaaaaaacagttctgaaatggtctgtttatcactcatttaatcagacggaggtaaagcatataatagaagtctacaaatctctgagacacttcggttacggcagccgtacgatctgcacaagcgtctgaacgttgctgttgcatgcacacaccagacgctgctcagtcccacatcgcgctgttttttggggagaaacacagctccggacagcacggttggtagctccgcatctttagagctgtcacagagcgaactcctcactccacaacggcagtctggagcctcgctgggctatagcaggcttacacgaggggcatccgcctgcgtacaggtgttaacgcagcccgaactcgggctctctcggcatgaggggctgtgcccgcccctcagagcagagcgcggcgcgctgcctcagctcggtccccgcttcccgcccgccgcgtcagtagggggaaagaggggagagccgcggccgtgcccgtctcctttgcgtcccttcttccttccaggtcccgatggcgagttgcctctgggacaaaaaaaaaaaaaaaaaaaaaaaactgtgagtgaacggctttaaaaacccattggaaggtggcagccgggagagccgcgcgactcccgagcagtccgaagcgcgagcggggccggacgagggtccgctccgtcccgggaaccccagcgccgccgccccggcacgccgcggggaggagcccgccgcgcagcccgccgtgcggcccgtgggccgggcagcctcagggccgccccttcgcagggctgggtggggtgcagggggtggggtgggtgaaatagaggcgggcgcgtg

SEQ ID NO: 10 - DNA, 3001 bp, Gallus gallus
ctttatttaccattcaggatcccaaagtgctgctaactacacaagttaacagctgtctgctcacacagttcctggctcctacctcctcaggcatctttttcctgtttatacttgtacatcagatgggcctttggaaaaatccctgattttcttttgttctgttttctttgttctgtttccatgcagtcttttcccctttggagatgacatgtatgggaaatggcttattgcagcaataggagacagaagcaaaaacaactgcatcatgggtaaagcaaacatcaaagcaaagcacaagccaagtagctgtgaaaaaattatttcgtatgtgatatttcagtgtgttgaagtccagcagtggtagattttttggcaacacagaaaaaaataaaacacgtgcagctaccacttcccaaatctcgtttctgtctgcatttttactaaaagcagacaagactatggacacgacacacctgttgcagttggtgcgttgccatttctgccttctctaagatgtgacaatacattaaacctacctgtagggacggttttaactgggcttgaattggatcaaaacaataaaagtaagtctgaaagactgtgaaaagcaaattggaacccatctttcattaaatattaattacctgtttgacagcccacgtacaaaaattagcatgcttattgcagatgtattgttatcgtggatgtatgtgtattgttgtaggtagccaacatgattaagtacttttagcaattcctatctaaaggtaattaagttctgcagtgaaatggaaggaatctggcatacaaaaataaacattaaggatggcagagattttaagtgaataaacaaatataactatttccaacatacacattctcctttctcacaatttacataacgcaaacggctctgacaacatcttcatattctgtttatctttaaatagagctgagctaagaaaatctgcatattgtgtttctgcagcttcaacctcctgacccgctggagagtcctgcaggctactgagcaaggaaagttccctctgcgggtaaagagcccatggcaagcagcaagcagcgctcgcaaaagcagcaaacatgcctcatctaagtaagtgtatgtgtggagtttgttaaatatttatccagatttatatttgagcaataaaaattacatgctagaagtacttcaactatctttaagcagcaatacccacacttccgtagtttgaaagcttttatttttagtttgtaagagagaatctggtttcctaagagggttgctatgctgacattaattagaagcacaacagaactggctttggaagcaaactgacaatccaaatcaaacaggagaaggtatttttattatttatgttaataccaccattccttttccacctggaataacagagaacaaagcaaagcaactttgtcctctattcattgcaggtagctgaaaaattgtaagtgacctgacgagatccctctggtcttttatgagggtacggttttaattagataacgttatctcttgccccttgaagtcaggatctccagatgcatcacagccacctaagccttatgtaaaggcacacttacagcaatacaaaggtgcacatgagcgctggcactgcccggccctgctgtgttcccacagcagtggggaaagcttgcagacagagctaattcccactgaaggccgattactttcaagatgtaagatttgctggttccgttgtacctaaaatagacactgagattctctggaagcaatcacagcaatgggccagccttgctaattaggaaaaggcttttttttttttttttcagttggatggttgtctgctgaaggacatctctcaatgcagggatgattgctcgcgtgttcttaattgttgatgcttaattggattgtgtccagtccttccttccaagtgcttctacacagagtatatttaaattcattcgtgtctatttttgtccacattctgtgcatgtctggaacagagcaacaagttttaacggcagctttgtaaactctactgacaaacgcaacccttcacagaaacagctttcatttcttgttttattactccaccttctggttaactcacatatatttttcaagcaagatctggagcgagaatggaatcctatcgagcaggaaaactccctcacaaaagctacaaaagccttcaacggccgctcgttccgcaggccgtgccacgaggcgaaacgcctgcgccgggcacacagccctcggctctgcccgctgcggccagcagcccccgtcggcccccgtcggcactcttcggcagctctcggccttcgggagccaatcagagggctccgtgcgttcccgctttagctcgctagcgcgtgcaggctgaagagagttccgcagggcgggcctgagggaggcggcggcgacggtcgttggtcgcgggtttttgtggtccggggggcccggcgaggaggtagacgtgtggcgttgtggagacggcggtctcccgtttgtcgctggagcggcgtcgctttgtgactgaggtaatgctgagggtttggccgggctggttggggggaggaagaggatttgcggccgagaacggcggagtccgaagggatgggcgctctgtgaggagccggcagtctataggggccgtttgggtgtctgtgtgctacaaataaagcgtttgctgctccgctgataatgtagtgaattagtcgggattgtgttggtaagtgtcacttcaaagtggcgttggtgggctcagagtttgaagttcctgccatgctctccggtgcagctgtttttcttcacagcttgagaatgcctgggctgcgctgttaggcttcagcttggctgtaaggagctgttaattacccacactggatgggcaaatgaaacagtgcttcttgtaacacgtttgagtaaggatgggggaagcagaagactgagcactaatgctgtgggtgctttgtcttcaaagggaac

SEQ ID NO: 11 - DNA, 3001 bp, Gallus gallus
tggacagattcagctgaccagccagaagggctgcttcgtttgccaacacataagcatctggcatcatctggggggccgtgggctgcctgagacgcccacacatgacgtgaaggagtggcataggatatacttgtataacagctacaaagcagtagtatattcttagtgtatctccaaagagatcagaagcatcttttcaggcgaagtccagatctccacagatacaaaatgagtgtgcagctatatctgtagatgctgaactttgtggctctgaagctgcgtcctgcctttgctcacgggagatgaccacagcctactcaggatcacagggaagttttgcctcttttgttgctttctttgaactgaggatgatcaggaagaccctgaaatgttctaaagctgtattttgtatgaatttactgttaagtgttactttgtccccaaaagagtctttctttctgagtagagtagattagaatagaatagggtagagtagagtagagtaaggtagagtagagtatcaagtcttggctctggtctttatcttttgggtgatcttgggcaagtgtttcaaagatggaaagatgtactcacctcccaggagaacctcaacccatctgttctgtgtttctcagagcatgaaatgcatcatctttctaggacaggtgaggaggggcctctccattgcctcatcttccatttagcaacaattaaataagaagttagagtggaaaggaatactttgaaggacatgcaggagcttgactgagatctactgtaggcatcacctctggtggtcattttctcccttcagcttctaacatgagggaggaaacaagcactgagggctctacaaaacttgcctaacaatagtttattcctgatttctcaccacacatggttgctcctatggctgcatgagattaaagactccgaagtcttaagtgatcaagaggccttcaggtgcaaagtgaattgtcgtgtttgagatgcaagcaataggctgaccagcaaagccaggaagagaacagaggcgaggattcatgcctccacactaaaaaaatagggtcattttctgctattgtgtttctctgctcaaatagtctctataacttaagagttaaacctgtagaaaaaaaaggcatgatgaaataactgacaatgtttgaccttcctgaattacatctaggagaccacaaggcttaggtcagatgcagaagccttcattttgattcagaccgaaattttgcaaagcgtcttggtgtctgaggaacataaacgtcattggtttcatggtctatattggtatattatcgagtgctgaagtacaagtacaggaattcagtggtttctgctgttttgagcactgttgatggcccagttttcacctgtgtcagctagcagggatcctaacagttcccaagctcagtttgggaactggcacacccaggggccattcctaggatggagattgaagatgaccatctatttaagaagatgagtgaatggaacagtcattccttttccccattggtttgagggcaagaacactggccctggcactatttagtgaactaatagtgatgcagacaatgaaacctggactcctggctgccaaatcctctgaggagtatgacagcaaggaatataagctgtagtgactgaagctagctggctccagtactgcctccagcagcacagccagctaaatgatgcaactagccttgaagttgggcagctacatgggcatggatttgctgaattactaggtctgcctgctagcagtaccactcatagccaggttaaaactaagtagtgagcagtgtagacatgtccttaagcttctacacagatcaagtttgttgagggcatttttggggaccagaattacggcagtttcccatggaaaataagcatctgaagacctttcaaaacttgggccataatgttaaccacaaccctgtcaccatagagtgtaaaatgttttcctggataccttcctaaaataaagtaagactgatggaatgctgcttaagttttgcgtattaagcttagagaggaagaaggtaatttatggtttgggctcttcaattccttggaatagggtattcaggatatctagggtatctctaggctgtcctgtgtgggagaaaggcaactggttactgtcttgctgctcacaaaaaatacattgaactcattgccacacagcatcattagagtaaagacaaggacgagggatttgtgcatgtagagagagtaccttgagctataataacacagatataaataaagaagcccactgtttttggaaagactttgtgcctgctttgtgccatgcaaagttccatataacccagccagtccagtctgcctactgtgagtccagtaggctcagagtaacagagcgctggctttgtcttgattgatctgatagaaccattcctacatctttatcattaattttaatgccattactgaggaccttattatgaagcactgggagcagggaggagacttttgtcactgatgtgaagtctagattgcccatgcatactcattgtcactccttgtagtacattcctctccattgttctgttattctttgcccaccagttgagaatgggtagcaattaccaagcctatttgggttgcagtgattagccaaatcccattgaaaagatgtcccaaatcccttttgggacagcgcaaatactactttgtttaggaagaggccacattcagataggtgatttttggagaagcacatgcaggatatatctttgctgtcatactggcacgaattcaggaatttgtaacattttaagtatttgcagatccctctgtgctttccactgccacacagaatcctgtagaatgtatatacactgcctgactatagatagctttgtgaattaagtcaaattcaagtaaaatgcaagttaagagaacaatatttgattccttgcagtagctggaagcagaggacactggtgatgtc

SEQ ID NO: 12 - DNA, 3001 bp, Gallus gallus
ccatgtgatgcttgtgctgtgaggcacgtgtgcctgtgatctctttgtcagcagtacccgtctgttgtgtacaggatgatcctctccatgcttcgcagcgcaggaagtataacctgccactctcttctcagcttcatttcaaccacaccctgaggaaatgagatgaagaatcagtgtgaaataggtataaagtcaacagcatttaaagcaactgtacagatactcttcctctgctgaatgcttggttgtacatgcactctgtatctgttcttgcaaagcaaagcccaagtaatccaaaatagccacagaaacatcactgcagtaaatatagcactcaaactggaagctctgaagagacctattctttacttaaaattatgagttacaacaacagaggcttccaaacaggaattaaagatggtgtatttctaaacgctgccataagaaattcctgtggttgtaggggaagggagcttgtctgcaatcagagtgaggaattcacttcaagtcttttggtggagatcatgccatatccttcttctcctccactctggccgcacaacattgagaagagcatctcagaatactgttctttgcaaagacttgtttctggcccacaacatctttttggacacaactatcttcgggcaaaatactagaaagataatttgagtttggtgccagcaacagggaacatatccttgcagaatcagaggccagaagggacctctggagatgatctagtctaacgccctgctaaagcaggttgaagctgctcaagggctgcttcactgcaagacaattctgagcagaattcaccagctggtcatggctttctcctgacttttccacttaggtgagctcatgcacttacgagcattcataggcaattactggcagtctctcagtattgcctgtccaagtctcaccatctgttaccgctctgcccaaattaaccactgcgcatgccagtcctcaaatgccttttcctggggtcagacacaacctagatgcacactcacagattatgctgtagctttaggtaaataccagcaaaattagcgtttccacaaattaaaccaatgacctgaggaaagtttccgtgttttgagtgggtcaacaaggccagcaccaatgcctgacagccaatgcacagctgttataccatacaattttttctcacttatacttgttttgtcttactgtatacaaaacatggttttgaatactaattattattactttagctgttccgtaaaggttaaaacaaacattctgttctgtccttccggatcaaagagcctctacagtaacagtggagtgtttccatgagcttgctgctctgcctcagaacagctgggagatacacttctgcatgtgtagggctaatcaatagctgctctccttggggtggatcgtttttcagcactgggtacagtgaaagaacaaacttgtacagagtccaaaacatctacttaaaaaaaaaataaatcgatcttcggggtaatgacagaatgtgactaacaattcttatctttggacttaaaaacatgtaaggaagtagaaaacgagggctgcagattgggaatttttttgcaaaaatggaatgatagaacaacagaagaccccaccatggtgacagcccatctgagctgccacaggcgtggggaccgtggacccgaccgccaactgacgttctgtcaccttctgagatgctctcagctttgctgcagggggagctttgcctttcattttaaaggagtcagaatgccaccagcacttataactgcagaacggaggttaattttagcccacggtaaattaaaagtatccataactacctgaatcataaagttaatgaaatcacacagctgaagtgctgcagtacttctgtatcggagaaagaaccgttcttcccagcttaagtgatagtccaaacccagactccggcagccttgagagacattaccaaagccatcctgctcttccacatgcccaactctgccatggctatctgccctaagtaacacactcttattgcacaagcaattggactgcagtgcggttacgtgtgccgcacaatgctactcatgaggcagcgtcgatctcttcaaaggctttagaattaaggtaagctgaggctcgagttcaaagaagaacctgaaacgcaaatgtatacatctgagccgacgtgcaaagagtcacttactgtaactcaggtatgccactgcagagccacggccgcccgcagagcgctcccgccggctactcggccaggcctcgccctcccgctcatccccgtgcttcaggccaagctccggcagccgtgcccgagccccgcccgacacctcgcagctccgcggcacgactcgccgatgcgggcaccggccccagccgcacagcccgtcctgcgggcgctccgccggggttctgacagaacagctcccgggcggcgtcacgcgcccgtctgccggcagaaggcacgcggcggcagtcccggcgcgtcgctgcggggcgaaggacgcagcgctccgcgagccgcccgacgggcccggagccttttctcgcagctccgacggggcggttcgctctcgttccctccgcttccccgagctccgccgacggccgcgggccgctacccccccctgcaggcggccgcgccgctccgcagcctcgaccccatcgctgccctcgccccgccgccccggctcgtcgcgctgccccccggctcccggctccgtcgcggacgcccccgcggtaaccgtgcggccgcggccgtcccgccgggcgaggcgcggagctccggtacccgcgggccgctcggcccttcccccgccgccgcgcgcagctccccatcccccccgcagggggccgaggctccgggggctccgtcagccgcggccccctccccgccccgccgcgccccgccccggcccgccgggggctccggcataaatacggccccgagcggcgctcccggggcggcgcggctggggagggg

SEQ ID NO: 13 - DNA, 3001 bp, Gallus gallus
aagaacaagatatttttctgaaagaatagaaggactttgagaactgatataacagaaaggagctgaaatcaataggaaaggaagggccaatcatgcatttagaaattaataataagtaactctaagacctcaactcttaaaaatagctggagaaagtaacaacctcggtatattatggcaatgagctataaatttaatacagatgttcttaaaaatgcagatcctttcattgacgtatgttccatgttataccaccatagactaaaaactctattgtttgctaattttggagagtaatgatcaggaacaattatgactttagaaaagatttagaaaaaagctgagggaaaaggagaacctggtagatgagagaaaatattataatgcttatttgcctagaataagataaggaaggctgaggcagaaagggagtcaactgattcttttgtatcctgggatgtaaatgattaactgtcagaaaagaaagaaggtattcaagataaaaaggtattcaaacataagcatgtgaatagttgtacatagacttgctagtgaaaataagttggaagttagatctttacaaatgttaaaacacagagtaagtgtcaaaaaaattttgagaagacaaaaactgagatgcttctcagatggtctctgttctagttatagaattaaaggctgcacttgctgtgccagcagctgatgatgcaataagccaatcagcagatttgccctgttttctctctgttggtttacttattgttcctccatataatgtcagagactactttatgacctgattagtaaaataaggaaatatgttaaatacaagggctagcatttcacaggagaatgacttcttgctctcaccagcacatatttttaaccaatctatttgcagtcattttgaaataacaaaggaacaaaaaaattcagacagaaaattaagtgttctaaatatgatggcaagtaaatacatacaatggtggcttattggactctgtacataagttggaagagactgtcacattcagtgtattttttctatacaatctaatgtcaaaatatatctatcatgctcagttgctagtaatagttcatgcttttaaaaattcagacagatgaatgaaaatatttaacattgtgtaaaggggaaaaaaaggctttagttcactgaatttttgaacatatgtcaagttcagcaaccacacttgaatgcaactaagcctcttcagtgttgcctgtcttttatcttgggcaaggactgttttgggactttatggctgagcatcccacaagaatcctgcaacagttcttgtaacaaaatgggaagctgaaatcgaagtgcaagacctcttagttcctctcattttagactactcagtcaggttgcagaatcctatgaattacattcttcagtgtctgagtgcttatacattatatgacagtgccaagtggttaaggacctattcaggaagctatggagttattaagttaagcaataaaatatagacattattaaagttattgaactgattatgtgatatttgagtcttagcaattagcattcagttcaggctttatttaattaagtaaatatactgccatgtgacatccacttgtttttgttcttaaaatttgtattattccaaaaatgacaaaaagaaaaaaaaaaaaaaaaggaactgggtgtaggtattatgctttagcatgttaaaattattattctactttacatttaatcccatattttgttataggacgggagtttggagcataggagggggggttggagcaaatattcctccccccccccaaaaaaaacaacaacaacaacaaaaacaacaaccaaatgaacaaacaaacaaacaaaaaaaaaaaccagcaaacaacaacaaaaccaacaccaacacagttaaagctagtgggaaggttcagtgtattatttgctgatttggaaaagacttccatacaaaacatgttccaaaatattgtacagaataaaaagctttgtgtaactacagcacaaaaacacaatccaaaatgctttggctataaaaggaagccttctgtttttcttacatctgaggccaaagcaattttaatttctgacttcttaattaatgttaaacacagttcagattgagtttctcatcaaacaagtattgcaacatttgtagaattacagttattttgttcaatgtcttatctttaatttcatcatcctgagtatgagagctgttaaatttaaagattatgccatctgtcaatggaaatctgtgtttcagagctgtgaaaagcatggctgatagttttgttgttttactatgatttggccacttcttattctattctctttatttgtgctgtgctgatgcctagaggctctaagtgggataataaaaaataagaattttgctttatgatagaattgagaggttttgaacatagtttttatcacataccaacatagactgagaagattttgcaatgttttgtgagaaaaggaagaggctggcaggctgggatatgagattcaagttcatctgtatcttctgtctagagacctgaacatgtgttagggcttaggtaagactccaaaccactaatggcagttacccctgtccttggtcaatgaactactattccttttgtcactgtaagagactattcttttatataattgactcaaagtttgctgaggaacaagtccaggcaagtcctgggcaaaggcagagaaatcttttgtcttgaggacactgatggacaggtcctggctaaggattgtgaaatcctctaaggagcacagatggacaaggccaggggcatcgagagagagataagctgctgctaatggccgggaagcggtctttttgtgtggacttatctcaaggaaaatggccatctcaggaggtatgcacaggactcttgctcaagcacccaggaatgtcacgtaggcagcagaaaatggagaataaaagagggtccaataaccacaacg

SEQ ID NO: 14 - DNA, 3002 bp, Gallus gallus
ggtggcagggagcgctatctgctgctctcgagcagatcctcggggatggggacgtccctgcgtgtgtgcaacgtctgccccagttctgcggcctctttgaagccgtaagaaatcggagctcgaagtatcaccgcacagaaccctcttctttcaaacagaggtagaagaagcttcaaattattgaaaatcagtctgtgtgtgtgtgtgtttttttttttaattaaatgtacatacatttaaatagggaagtataaaatattccaggtcttgctccttgtgctaatcaaggctgctggaggctccccgagccatcatcttttataattataattacacacgtcaatcaagaccttcaggcaaagtcagaacaaggcaggcaaactcaggagagaaatcacatttaaaaaaaaaaaaaaactttggagaacttggtaaggaatggagaagagattagaagaaagggtaattaaggttgctcaaggcaatcatgagtgctagaactccatgttaaattaaacgtagccacgagggaccaggtatcatttataatgtgtactgccctaaactgttacttcggagaaacgtggatgtgtaataagctctccaaagtacatacatctgtagcgatggctggcggagcagcactgcagcgcacacttcgagcttgcccaagccttccccggtaggacgacacggagcccacgagtgagaccgcagctcccggggagggccgccagggccgggcccaggtgcgggcgtttcggccgttctgccggggctgcccgcggcccccttccccggctttcgcacaaatcgtgggtgcctctctcggagcagaacccacgagagcagcggcacggggcgcctcggcgggacttcgccgggcggcgtgaccgagaggggaggggagggagcggcggctccccgacggctcagcgggccgccccctcgccgggcccaaagccgcggcccccggcccgagcgcggagcttcgagccacgaacctcagggcgggcgcccgaaggagcggagccgccgctgcgctcggccccggccagagccgagccgagccgcagccgggagccggcgcgggtgactggggcccggcggagatgctccccggcggccgcacggtctcggcccggcccgctcctacccagaggacgggcgcactccctcgggggctctcggccgccccgcagagcaccgcgctggcgcgtcccgcggctcgtcgcgcctctacatcgcgctgcaggagagctgctaaggtgtgaagttacagcttgagaaataacgcgatgacatagaccaaaccttccctgcctgtgctccgtcccgaagggggatggcaaaatgtaaggagcccgtaagctccgagcctcactgagtgataggtccagccccacaatgctgaacaccctgcaccgaatgggaaagacagacaaaattctgtctgcgtagggctggctcggggtccgcactcagttaagcaaagctctcgccgtcggtgggtgagaacagttcgaactgtggactaaatggcacgcgcgatgcactccctcccccctccccctccgctccctttcgttcatcacagcggctctaagttcgaacacgggtccgctgctccgaaatctgtcccagccggtgggaaaagaggtccccagcgccgcacagttccgtgcctcgctttcaatcggaagcagttttcagcgccgaatatttttcagcttttaatatacataaccgtgactgaactctcagagcagcacgggcggggggtctcggatgctcgcagaggtgacaatagcactcagcccacaccgcccggtgggggcggccggagcccgcggtgcccgcagcgcggccgggacgggtggggcgggacgggcggaccccaccgcccccggggctgcttcgcaccgcagggccgcccagctcccgctgagacacccgctggagagaaacgagttatctggggtaacgcggggagtagtgccgctcaccgcaccgtgagccggggaagggaagctcttgctcaagcgatggctcggccagggcatcggattttgtgcaaacgatgcagccggcaccgatagatttcgcaagaagttttagggaaacgagacgcaaagagccagaatacataaggcgagcttccgcgcgctacgttcaggttctggggcagagaggcggcagcgggtcccgagggccggggttccccagacgccgaggttcccgctgcccgcccgcaggaggacaggcgagcgccgggggtcccgcgggaggcagacgggagctgcagtggggtgcgcggcgggctgggggccgtcgggctgctctagggcgggcgggttcggctgcagccccggccccgcagcccggccaacaagcgggcggggggcgatcacgctgtgcaagcgtgtgcgagcgtgtgtatgcgcgcgtgtgtgcgcgggggtgtcgctcatttctcctcctccgtattggctgcgagagccgcgtcggtgcccccccttcgccggagccccccgcggcccccgggctccgggagcgcctccccacgggtccggggcgagaggtcccggctcggcacctccgcacttgtggcgagggaaagggggaggaggaggaggagggaggaagggaggagggggccgggggtgggaaccctgtccagcagcaccggcggcggcagagcgctcccgtgcactgcgcctctccgcatggatcggggcgccgggctgcagccgcgctgagccccgggcggagctgctgcgggctcccggggtcgctccttcctctccgacacccccggtcttcctcctcctcctctcctctcctctccgcccccgccgcctgcaaaccgacaaccgctccgcggctctcccggccgcgggcacccggcggcgcttcccggcggccggggccgctcggcgctcccgctgcagcggggcaagggctggcgcggagcggagggagggaagaggaggaggaggaag

SEQ ID NO: 15 - DNA, 20 bp, Artificial Sequence
  Synthetic
tctagggaaagagagcactg

SEQ ID NO: 16 - DNA, 20 bp, Artificial Sequence
  Synthetic
ggtctcggcgcaccgccgcg

SEQ ID NO: 17 - DNA, 20 bp, Artificial Sequence
  Synthetic
gtacacagagagctacaatg

SEQ ID NO: 18 - DNA, 20 bp, Artificial Sequence
  Synthetic
aatcataggattgctcaggt

SEQ ID NO: 19 - DNA, 20 bp, Artificial Sequence
  Synthetic
caacatccagatctaccccg

SEQ ID NO: 20 - DNA, 20 bp, Artificial Sequence
  Synthetic
ggcgagacattagaagaacg

SEQ ID NO: 21 - DNA, 20 bp, Artificial Sequence
  Synthetic
aatggcctcaagttgcacca

SEQ ID NO: 22 - DNA, 20 bp, Artificial Sequence
  Synthetic
gaaactccgggttaacccag

SEQ ID NO: 23 - DNA, 20 bp, Artificial Sequence
  Synthetic
gggggggggggggaaggaaa

SEQ ID NO: 24 - DNA, 20 bp, Artificial Sequence
  Synthetic
actgaaaatgggcttcaccg

SEQ ID NO: 25 - DNA, 20 bp, Artificial Sequence
  Synthetic
actcgggaggcagaggcagg

SEQ ID NO: 26 - DNA, 20 bp, Artificial Sequence
  Synthetic
gatttcttgaaagagaacag

SEQ ID NO: 27 - DNA, 20 bp, Artificial Sequence
  Synthetic
gagaagggggaagaaaactg

SEQ ID NO: 28 - DNA, 20 bp, Artificial Sequence
  Synthetic
aggcttcaatgtggaaacag

SEQ ID NO: 29 - DNA, 20 bp, Artificial Sequence
  Synthetic
ggtggcggcggcggcggcgg

SEQ ID NO: 30 - DNA, 20 bp, Artificial Sequence
  Synthetic
guaauacagagcuaaaccag

SEQ ID NO: 31 - DNA, 18 bp, Artificial Sequence
  Synthetic
gcggagggagatgaaaca

SEQ ID NO: 32 - DNA, 20 bp, Artificial Sequence
  Synthetic
aagggatcacacagctcaag

SEQ ID NO: 33 - DNA, 19 bp, Artificial Sequence
  Synthetic
tcccatggcattgatctgt

SEQ ID NO: 34 - DNA, 21 bp, Artificial Sequence
  Synthetic
ctatccacgcccattgatgta